(19) United States
(12) Patent Application Publication
(10) Pub. No.: US 2010/0136517 A1
(43) Pub. Date: Jun. 3, 2010

(54) MATRIX STABILIZATION OF AGGREGATION-BASED ASSAYS

(75) Inventors: Grace Y. Kim, Cambridge, MA (US); Christophoros C. Vassiliou, Cambridge, MA (US); Karen D. Daniel, Newtonville, MA (US); Michael J. Cima, Winchester, MA (US)

Correspondence Address: CHOATE, HALL & STEWART LLP, TWO INTERNATIONAL PLACE, BOSTON, MA, 02110, US

(73) Assignee: MASSACHUSETTS INSTITUTE OF TECHNOLOGY, Cambridge, MA (US)

(21) Appl. No.: 12/599,109

(22) PCT Filed: May 7, 2008
(86) PCT No.: PCT/US08/62838

§ 371 Date: Jan. 6, 2010

Related U.S. Application Data
(60) Provisional application No. 61/041,446, filed on Apr. 1, 2008.
(60) Provisional application No. 60/916,408, filed on May 7, 2007.

(51) Int. Cl. C12Q 1/70; G01N 33/544; C12Q 1/68; G01N 33/53

(52) U.S. Cl. 435/5; 436/528; 435/6; 435/7.1; 977/920; 977/773

(57) ABSTRACT

Methods and apparatus for stabilization of aggregation-based assays are described. In various embodiments, anti-analytes are dispersed within a matrix. A solution containing analytes brought into contact with the matrix, so that analytes may permeate throughout at least a portion of the matrix. In some embodiments, the anti-analytes and analytes are mobile within the matrix. As aggregates form and increase in size, the aggregates become substantially immobile within the matrix. As a result, signals representative of an amount of aggregation within the matrix can remain substantially constant. In various aspects, matrix-stabilized aggregation-based assays provide for reliable quantitative analysis of analyte concentration with test solutions.

### CROSS-REFERENCE TO RELATED U.S. APPLICATIONS

The present application claims priority to U.S. Provisional Application No. 61/041,446, filed on Apr. 1, 2008, and to U.S. Provisional Application No. 60/916,408 filed on May 7, 2007, which are incorporated by reference in their entirety.

### GOVERNMENT FUNDING

The work described herein was conducted within a research program supported in part with funding from the National Cancer Institute, Grant No. 5-U54-CA119349-02. The U.S. Government may have certain rights in these inventions.

### BACKGROUND

Aggregation assays are widely used in the fields of chemistry, biology, and medical sciences to detect the presence of a suspected analyte. Generally, the assays are easily performed, e.g., by adding a solution suspected to contain an analyte to a solution having a known anti-analyte. The presence of analytes in the mixture induces the formation of aggregates as multiple analytes and anti-analytes bind to each other. Depending on the system involved, the formation of aggregates can be seen with the naked eye, or detected using indirect means, e.g., optical scattering, optical absorption, or fluorescence, each of which may increase or decrease as aggregates form. Aggregation assays are commonly used in the art to provide qualitative information about an analyte, i.e., whether or not an analyte is present in a sample without regards to the amount or concentration of analyte present.

### SUMMARY

As described herein, the inventors have solved certain problems that have previously prevented quantitative aggregation-based assays to be performed on a reliable basis. The inventive embodiments that are described herein therefore relate to methods and apparatus useful for aggregation-based assays which can provide quantitative information about analytes in a sample.

In various aspects, inventive methods for aggregation-based assays include steps of (a) providing a matrix, (b) contacting a test solution suspected to contain an analyte to the matrix, and (c) detecting a signal representative of an amount of aggregates that form within a volume of the matrix, wherein aggregates larger than a certain size are substantially immobile within the matrix. In various embodiments, the matrix comprises a substance having a viscosity greater than about 1.5 centipoise and anti-analytes dispersed within the substance. In various embodiments the anti-analytes and analytes are mobile within the substance. In various embodiments, the test solution is contacted to the matrix so that analytes from the test solution permeate through at least a portion of the matrix. The analytes may then bind with anti-analytes within the matrix to form aggregates. In various embodiments, aggregates of anti-analytes and analytes reaching a certain size become lodged or suspended in the matrix, and do not precipitate out of the matrix. A signal representative of an amount of aggregates which have formed within the matrix may then be detected. The signal may be detected by any one of a variety of techniques including, but not limited to, nuclear-magnetic-resonance (NMR) imaging, nuclear-magnetic-resonance spectroscopy, nuclear magnetic relaxometry, optical scattering, optical absorption, optical spectroscopy, optical imaging, optical fluorescence, infrared imaging, infrared absorption, infrared spectroscopy, infrared scattering, X-ray imaging, X-ray absorption, etc.

In some embodiments, inventive methods for aggregation-based assays further include steps of (d) measuring a value of the detected signal, (e) comparing the measured value with calibration standards, and (f) determining a concentration of the analyte in the test solution from the comparison. In various embodiments, the step of determining a concentration (f) provides a quantitative analysis of an aggregation-based assay.

In various embodiments, an apparatus for a matrix-stabilized aggregation system includes a coverable vessel in which aggregation of analytes and anti-analytes may take place. The vessel may contain an amount of matrix, throughout which are dispersed anti-analytes. The vessel may be adapted for the introduction of a solution containing an analyte, e.g., space may be provided in the vessel for the addition of a solution suspected to contain an analyte. In various embodiments, the matrix within the vessel comprises a substance having a viscosity greater than about 1.5 centipoise, and within which anti-analytes and analytes are mobile. In various embodiments, aggregates of anti-analytes and analytes larger than a certain size are substantially immobile within the matrix.

The inventive methods and apparatuses also provide for in vivo matrix-stabilized aggregation-based assays. In various embodiments, an apparatus for in vivo or aggregation assays comprises a vessel containing a matrix and anti-analytes, wherein at least a portion of the vessel permits the inflow of solution containing analytes. The solution containing analytes may be native to the in vivo environment. In various embodiments, the vessel is adapted for in vivo placement, and the matrix comprises a substance having a viscosity greater than about 1.5 centipoise. In various embodiments, the anti-analytes are dispersed within the matrix, and the anti-analytes and analytes are mobile within the matrix. In various embodiments, aggregates of anti-analytes and analytes larger than a certain size are substantially immobile within the matrix.

The foregoing and other aspects, embodiments, and features of the present teachings can be more fully understood from the following description in conjunction with the accompanying drawings. All literature and similar material cited in this application, including, but not limited to, patents, patent applications, articles, books, treatises, and web pages, regardless of the format of such literature and similar materials, are expressly incorporated by reference in their entirety.

### BRIEF DESCRIPTION OF THE DRAWINGS

The skilled artisan will understand that the figures, described herein, are for illustration purposes only. It is to be understood that in some instances various aspects of the invention may be shown exaggerated or enlarged to facilitate an understanding of the invention. In the drawings, like reference characters generally refer to like features, functionally similar and/or structurally similar elements throughout the various figures. The drawings are not necessarily to scale, emphasis instead being placed upon illustrating the principles of the teachings. The drawings are not intended to limit the scope of the present teachings in any way.

FIGS. 1A-1F represent embodiments of a matrix-stabilized, aggregation-based assay systems.

FIG. 2 is a flow diagram depicting an embodiment of a method for a matrix-stabilized, aggregation-based assay system.

FIG. 3 is a flow diagram depicting an embodiment of a method for a matrix-stabilized, aggregation-based assay system.

FIGS. 4A-4B depict an embodiment of aggregation where the anti-analyte 400 comprises a nanoparticle 410 chemically functionalized with a targeting ligand 420 which binds to a receptor 440 on an analyte 401.

FIGS. 5A-5B depict an agglutination system having analytes 535 with multiple types of binding sites and two types of anti-analytes 510 and 520.

FIG. 6 is an illustrational graph depicting a dynamic range 610 of an aggregation signal. The aggregation signal is plotted as a function of analyte concentration.

FIG. 7 is a plot of experimental data which demonstrates diffusion of an anti-analyte through a stabilizing matrix. The proton (¹H) transverse relaxation time T₂, as measured by nuclear magnetic resonance within a remote measurement volume 180 of the matrix (FIG. 1D), changes as anti-analytes diffuse into the region.

FIGS. 8A-8B are plots of experimental data for non-stabilized aggregation systems. The proton relaxation time was measured after intermixing solutions of analytes and anti-analytes, for various concentrations of analytes.

FIG. 9A is a plot of experimental data for aggregation carried out in solution. The measured T₂ value after the addition of an analyte is initially low, about 38 ms, and then increases over a period of time of more than about 36 hours.

FIG. 9B is a plot of experimental data for aggregation stabilized in a matrix. About 12 hours after the addition of an analyte, the proton transverse relaxation time T₂, measured via nuclear magnetic resonance, reaches a substantially constant and stable value. The analytes and anti-analytes correspond to those used for FIG. 9A.

FIG. 10A is a plot of experimental data for aggregation carried out in solution. The measured T₂ value after the addition of an analyte changes over a period of time more than about 12 hours.

FIG. 10B is a plot of experimental data for aggregation stabilized in an agarose matrix. The aggregation constituents corresponds to those used for FIG. 10A. About 7 hours after the addition of an analyte, the transverse relaxation time T₂ reaches a substantially constant and stable value.

FIG. 11A reports measured values of T₂* for non-stabilized aggregation assays carried out in five different buffer solutions. Each of aggregation systems shows substantial variations over time in the measured value of T₂*. (See EQ. 1 in text for definition of T₂*.)

FIG. 11B demonstrates stabilization of a signal representative of an amount of aggregation, e.g., T₂*, for matrix-stabilized aggregation systems using the different buffer solutions reported in FIG. 11A.

FIGS. 12A-D report data collected from dynamic range and stability studies. Dynamic ranges were assessed for five different buffers in non-stabilized aggregation systems (FIG. 12A) and matrix-stabilized systems (FIG. 12B). Stability for one of the buffers (PBS) was assessed for various incubation times, i.e. times elapsed after intermixing analytes and anti-analytes. Matrix-stabilized, aggregation-based assay systems provide stable dynamic ranges.

The features and advantages of the present invention will become more apparent from the detailed description set forth below when taken in conjunction with the drawings.

### DETAILED DESCRIPTION

### I. Introduction

Aggregation or agglutination of analytes and anti-analytes is a fundamental process which can be used to provide readily-detectable signals indicative of the presence of suspected chemical or biochemical constituents. Generally, a test solution suspected to contain a particular chemical or biochemical constituent, generically termed “analyte,” is combined with a solution containing a known anti-analyte. The anti-analyte can be any suitable chemical or biochemical component known to bind with the particular suspected analyte. In various embodiments, the anti-analyte and/or analyte will have plural binding sites, so that multiply-bound networks, termed “aggregates,” form from the analytes and anti-analytes. As the agglomeration of analytes with anti-analytes ensues, the aggregates can alter the appearance of the mixture, or alter signals used to probe the mixture. For example, light may be propagated through the mixture and the presence of aggregates may increase scattering of the light. As another example, magnetic fields may be used to probe the mixture using techniques of nuclear magnetic resonance, and the presence of aggregates may affect the way in which the magnetic fields interact with the mixture. Changes in appearance or probing signals can then indicate aggregate formation and the presence of a suspected analyte.

We have discovered that in some instances, unlimited and uncontrolled aggregation can be undesirable, since large aggregates may become unstable in solution and precipitate out of the solution. Any information that the aggregates could have provided through their interaction with the optical or magnetic fields while suspended in solution is then lost. In some cases, instability and precipitation of aggregates can yield false-positive or false-negative results for a particular aggregation assay. Such undesirable effects can occur when anti-analyte or analyte sizes are large, aggregates are large, or analyte concentration is high. In certain embodiments, aggregation instabilities may be tolerable, e.g., visual detection of a precipitate may be sufficient to conclude the presence of an analyte. In some embodiments, aggregation instabilities may be intolerable, particularly for embodiments of aggregation assays designed to provide quantitative information about analyte concentration.

The inventive embodiments described herein provide methods and apparatus to control or limit the extent of aggregation in various aggregation-based assays. In various embodiments, a matrix is provided as a medium in which aggregation-based assays may be carried out. The matrix can controllably alter aggregation dynamics. Various embodiments of matrix-stabilized aggregation assays can improve the stability of the assays and extend their usefulness in providing reliable data. Matrix-stabilized aggregation-based systems can provide for quantitative analysis of analyte concentrations. In certain embodiments, matrix-stabilized aggregation assays may be carried out in vivo and in vitro.

### II. Matrix-Stabilized Aggregation

### II. A. Overview

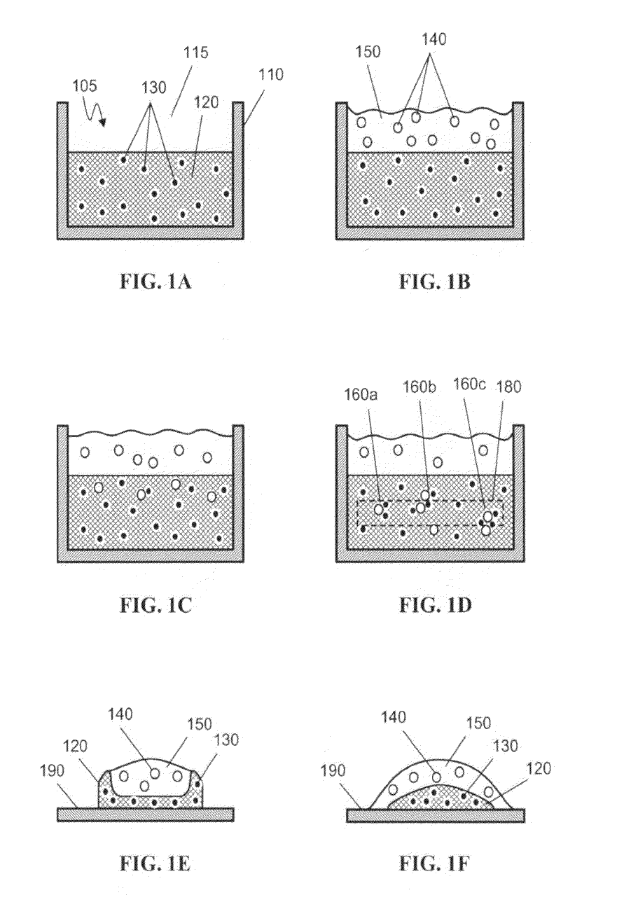

FIGS. 1A-1F, FIG. 2, and FIG. 3 represent embodiments of apparatuses and methods for matrix-stabilized aggregation systems. In various embodiments, these apparatuses and methods are useful for determining quantitatively a concentration of analytes in a test solution. In overview, a vessel 110 may be provided containing a composite substance 105. In various embodiments, the composite substance 105 comprises a matrix 120 having anti-analytes 130 dispersed throughout the matrix. The vessel 110 may include a vacant region 115 suitable for the addition of a solution, and/or introduction of pressurized gas or liquid. In various aspects, the anti-analytes 130 are mobile within the matrix 120. In some embodiments, the concentration of anti-analytes 130 within the matrix 120 is known. In various embodiments, the matrix is a substance having a viscosity greater than about 1.5 centipoise. A test solution 150 containing a concentration of analytes 140 can be added to the vessel, so that the test solution contacts the composite substance 105 and permeates through the matrix 120. In various aspects, as the test solution permeates the matrix, the analytes 140 move into the matrix 120 and become substantially dispersed throughout at least a portion of the matrix, as depicted in FIGS. 1C-1D. As the analytes encounter anti-analytes within the matrix 120, aggregates 160a-160c can form. In some embodiments, smaller aggregates, e.g., 160a, may have some mobility within the matrix 120, whereas larger aggregates become substantially immobile within the matrix. The matrix 120 thus substantially retains large aggregates in suspension, and can prevent their uncontrollable aggregation and precipitation from the volume in which they are intermixed. In various embodiments, one or more measurements can be made to detect an amount of aggregation that forms within at least a sub-volume 180 of the composite substance 105. For example, a measurement may comprise probing at least a sub-volume 180 with a probing signal which may be altered by the presence of aggregates within the sub-volume. The measurement can further comprise detecting a value of at least one signal, wherein the signal is representative of an amount or extent of aggregate formation within the measurement volume. The detected signal value may then be compared with values from calibration standards to determine a concentration of the analyte in the test solution.

### II. B. Providing the Matrix

Referring now to FIG. 1A, the matrix 120 and anti-analytes 130 may be provided in a vessel 110, which can optionally include a cover, not shown. The vessel 110 may be coverable, and may further be adapted for pressurization of its contents. For example, the vessel 110 or cover may include a port through which gas or liquid pressure may be applied. The vessel may be made of any material, e.g., various types of glasses or various types of polymers. The vessel may be a stand-alone container, or may be connected to multiple similar vessels in an array, e.g., a one-dimensional or two-dimensional array. Similar amounts of the matrix 120 may be provided in multiple similar vessels. As an example, the vessel 110 may comprise one well of a multi-well plate, e.g., a 24-, 48-, 96-, or 384-well plate. The shape of the vessel may be varied, having vertical sidewalls in some embodiments or sloped sidewalls in some embodiments. In yet other embodiments, the vessel may comprise a rounded depression, such as may be formed by the molding of a polymer or plastic. In some embodiments, plural rounded depressions may be disposed in an array in a piece of plastic. The volume of the vessel may be any value, e.g., ranging from a few microliters to tens of milliliters. In some embodiments, the vessel or the array of vessels may be adapted to be mounted in a centrifuge instrument, so that the contents of the vessel may be centrifuged. In some embodiments, the vessel 110 may have an optically-transparent portion through which light may enter and exit the vessel without significant scattering or attenuation of the light by the vessel.

In certain embodiments for in vivo applications, the matrix 120 and anti-analytes 130 may be provided in a vessel adapted for in vivo placement. As an example, the vessel may be round, elliptical, or oblong with rounded features. The vessel may be small in size, e.g., the size of pharmaceutical pills. The vessel may be sterilized. In various aspects, at least a portion of the vessel permits inflow of solution containing analytes, where the solution and analytes may be native to the in vivo environment. The vessel may have a porous or semi-porous portion through which the solution and analytes may flow. The vessel may be placed in vivo by different methods, e.g., ingestion, placement by catheter, or placement by surgical procedure. In some embodiments, the vessel may be retrieved at a selected time after placement.

In various embodiments, the matrix 120 and anti-analytes 130 are provided in an amount deposited on a substrate 190, as depicted in FIGS. 1E-1F. In some embodiments, the matrix and anti-analytes may be provided in an array of discrete small amounts deposited on a substrate, e.g., an array of microdots on a microtitre plate. The substrate 190 may be flat, curved, smooth, or non-smooth. As an example, the substrate may be substantially flat, but contain dimples which may aid in containing the amount of deposited matrix. The matrix and anti-analytes may be deposited on a substrate in a variety of shapes, including a dished shape as depicted in FIG. 1E. A solution 150 containing a suspected analyte 140 may be contacted to the amount of matrix and anti-analytes, as depicted in FIGS. 1E-1F.

In some embodiments, the matrix and anti-analytes may be deposited as a film on a substrate. The film may be substantially uniformly thick, and cover an area of the substrate. In some embodiments, the film may be limited to discrete active areas, within which aggregation methods are carried out. In some embodiments, the film may cover substantially all of the substrate. In certain embodiments, the thickness of the film may be any value in a range between about 10 microns and about 10 millimeters.

### II. C. The Matrix

In various embodiments, a matrix 120 comprises a substance in which aggregation of anti-analytes and analytes occurs. In various embodiments, the substance comprising the matrix has a viscosity greater than about 1.5 centipoise (cP). In some embodiments, the substance comprising the matrix may be a gel, a semi-solid, a substantially solid material, or a solid material. In various embodiments, the matrix 120 controllably alters aggregation dynamics, e.g., stabilizes aggregates in a mixture of anti-analytes and analytes, limits the size of aggregates, and/or immobilizes aggregates greater than a certain size.

In some embodiments, the matrix comprises an intertangled mesh of submicroscopic, polymeric molecular chains. For purposes of understanding, this mesh can be envisioned as a collection of spaghetti, through which liquid and small particles may move. In some embodiments, the matrix comprises a collection of microscopic or submicroscopic beads or particles. The beads or particles may have diameters in a range between about 50 nanometers and about 250 microns. In certain embodiments, the diameters are substantially similar, e.g., about 50 nm±about 10 nm, about 100 nm±about 20 nm, etc. For purposes of understanding, this type of matrix can be envisioned as a collection of sand, through which liquid and small particles may move. In certain embodiments, the diameters of the beads or particles in the collection may be spread over a broad range of values.

In yet further embodiments, the matrix may comprise a combination of mesh and beads. As an example, material comprised of polymeric mesh may be formed into beads or particles of any selected size, e.g., diameters with values in a range between about 50 nanometers and about 250 microns. The matrix may comprise a collection of the small particles, where the particles are substantially similar in size, or the matrix may comprise a collection of particles having a range of sizes.

In some embodiments, the matrix 120 may comprise a liquid or flowable material. The liquid may be a Newtonian liquid, or a non-Newtonian liquid. In certain embodiments, the liquid may have a viscosity greater than about 2 cP, greater than about 5 cP, greater than about 10 cP, greater than about 20 cP, greater than about 50 cP, greater than about 100 cP, greater than about 200 cP, greater than about 500 cP, and yet in some embodiments greater than about 1000 cP. The matrix may comprise a gel or hydrogel. In certain embodiments, the matrix comprises a liquid or flowable material for which the Brownian diffusion distance traveled for a given time interval and for aggregates within a certain range of sizes is greater than the distance traveled by the aggregates due to gravitational forces for the same time interval.

Generally, the matrix 120 may be any material selected to stabilize the aggregation of analytes 140 and anti-analytes 130. As further examples, the matrix may comprise an agarose gel, a dilution of an agarose gel, a polymeric material, a ceramic material, a porous ceramic material, or any solid or semi-solid material having microscopic or submicroscopic pores. In various embodiments, the matrix may be formed from agarose gel, acrylamide, polyacrylamide, cellulose, chitosan, dextran, ficoll, silica gel, or any combination of these materials. In some embodiments, various polymers that may be used to form the matrix include, without being limited to, methacrylate, polystyrene, polyvinylalcohol, polyethyleneglycol, polyurethane, polycarbonate, polyarylate and polymethylmethacrylate. In some embodiments, the matrix may comprise a mesh of glass fibers, a ceramic mesh, sintered ceramic beads, cellulose, porous scaffolds, or inverse opal scaffolds. In some embodiments, additives may be used to increase the viscosity of a liquid-like substance. The additives may include, but not be limited to, alginate, polyethylene glycol, glass fiber, carbon nanotubes, fullerenes, and any combination thereof. The matrix may be substantially solid or solid at about room temperature, e.g., about 70° F., and flowable when heated to temperatures above room temperature. In certain embodiments, the matrix 120 comprises a mixture of 1% agarose and water. In certain embodiments, the matrix is biocompatible. In some embodiments, the matrix is biodegradable, and in some embodiments, the matrix may be biocompatible and biodegradable.

In various embodiments, the matrix 120 contains pores through which liquid and small particles may move. The pores within the matrix may vary in size throughout the material. The pore sizes may be distributed in value about an arithmetic average pore size. As an example, an arithmetic average pore size, also termed mean intrinsic pore size, may be any value between about 50 nanometers and about 500 microns, and the variation in pore sizes may be distributed about the average pore size according to a Gaussian distribution where the full-width-half-maximum (FWHM) value of the Gaussian distribution is related to the average pore size, e.g., the FWHM value may take any value between about 10% to about 100% of the average pore size. The distribution of pore sizes need not be Gaussian shaped, and may be approximated as a Gaussian function or any other suitable function. As a particular example for illustrational purposes, a matrix may have a mean intrinsic pore size of about 200 nanometers with a 25% distribution. For this example, the average pore size within the material would be about 200 nanometers, and have a 50 nanometer FWHM distribution about the average value. In various embodiments, the matrix 120 may be characterized by, and selected according to, one or both of its mean intrinsic pore size and intrinsic pore size distribution.

As used herein, the term mean intrinsic pore size characterizes the size of pores within the matrix 120. If pores within the matrix have substantially circular openings, then mean intrinsic pore size refers to an arithmetic average of the diameter of the openings. If pores within the matrix have substantially elliptical, or eye-shaped openings, then mean intrinsic pore size refers to an arithmetic average of the minor axis of the openings.

In some embodiments, the matrix 120 may be swellable. For example, upon absorption of a liquid, the matrix may expand such that it occupies a larger volume than it occupied before absorbing the liquid. In certain embodiments, the expansion or swelling of the matrix may increase the mean intrinsic pore size within the material, as compared to its non-swollen state.

In various aspects, the matrix 120 is transformable into a molten or flowable state, and subsequently allowed to set, e.g., subsequently transformed into a substantially solid or semi-solid or gel state. The matrix may be rendered into a molten or flowable state by heating the material. In some embodiments, a liquid solvent may be added to the matrix to transform it into a molten or flowable substance. While molten or flowable, anti-analytes 130 may be mixed into the substance to disperse the anti-analytes throughout the substance. In various embodiments, the state change of the matrix 120 to a molten or flowable state is reversible. Cooling of the substance or evaporation of the solvent may transform the molten or flowable substance to a substantially solid or semi-solid or gel state. In some embodiments, the matrix may be set by cross-linking polymers comprising the matrix. For example, the polymers may be cross-linked by exposure to heat in some embodiments, or exposure to ultraviolet radiation in other embodiments, or by the addition of a chemical cross-linking agent.

In one embodiment, a matrix containing anti-analytes 130 may be formed by first heating a material to produce a molten material, and mixing this with a solution containing the anti-analytes 130. While still in a molten state, the mixture of molten material and anti-analytes 130 can be deposited into one, or more vessels 110, and the molten material allowed to set. In some embodiments, the vessels 110 may be wells of a 24-, 48-, 96-, or 384-well plate.

In some embodiments, anti-analytes 130 may be incorporated into the matrix material 120 while the matrix is in a solid or semi-solid or gel state. As an example, a solid, semi-solid, or gel matrix may be immersed into a liquid containing a first concentration of anti-analytes. The liquid and anti-analytes may permeate through the matrix during a period of time, and thereby effectively load the matrix with anti-analytes. For example, while the matrix is immersed in the liquid, anti-analytes may diffuse into and throughout the matrix. The concentration of anti-analytes loaded into the matrix Cₘ may be dependent upon a concentration of anti-analytes within the liquid Cₗ, and upon the amount of time the matrix is immersed within the liquid. The uniformity of the concentration of anti-analytes within the matrix may be dependent upon an amount of time the material is immersed within the liquid tᵢ, or upon an amount of time elapsed tₑ since the loading of analytes into the matrix. After immersion, the material may be removed and dried, e.g., subjected to conditions which promote evaporation of liquid absorbed by the material. In some embodiments, liquid removal may be accomplished by lyophilization. In other embodiments, the material may be stored in the liquid bath, and subsequently used wet. For example, the material with incorporated anti-analytes may be transferred from its loading bath to a vessel 110 in which an aggregation test will be carried out, substantially immediately prior to the aggregation test.

As another example, a vessel containing an amount of matrix material may be substantially filled and stored with a liquid solution containing a concentration of anti-analytes C₁. During storage, the anti-analytes may diffuse throughout the matrix 120. Prior to use, the excess solution may be removed from the vessel, and the vessel briefly rinsed with a cleansing solution to remove any anti-analytes not diffused into the matrix. The vessel may then be used for a matrix-stabilized aggregation assay.

In some embodiments, the matrix 120 and anti-analytes 130 may be deposited in a vessel or onto a substrate such that the surface of the matrix incorporates topography, e.g., holes, divots, pillars, or the like. In some embodiments, the matrix and anti-analytes may be formed and deposited as a collection of small particles or beads. The use of beads or the incorporation of topography can effectively increase the surface area of the matrix, and facilitate diffusion of analytes 140 in solution 150 into the matrix. In additional embodiments, the matrix material may be made hydrophilic to promote absorption of water, and therefore analytes, into the matrix.

### II. D. Anti-Analyte

Any of a wide variety of anti-analytes 130 may be used in the various embodiments of the invention. Generally, anti-analytes are selected for their propensity to form aggregates with suspected target analytes 140 when allowed to intermix with the target analytes. Additionally in some embodiments, the anti-analytes may be selected based upon their mobility within the matrix 120. In practice, there may be as many or more types of anti-analytes as there are analytes for which a test is made. An anti-analyte can be a compound, molecule, nucleotide, protein, antibody, antigen, virus, bacteria, nucleic acid, lipid, ligand, carbohydrate, chemically-functionalized particle, or any combination thereof.

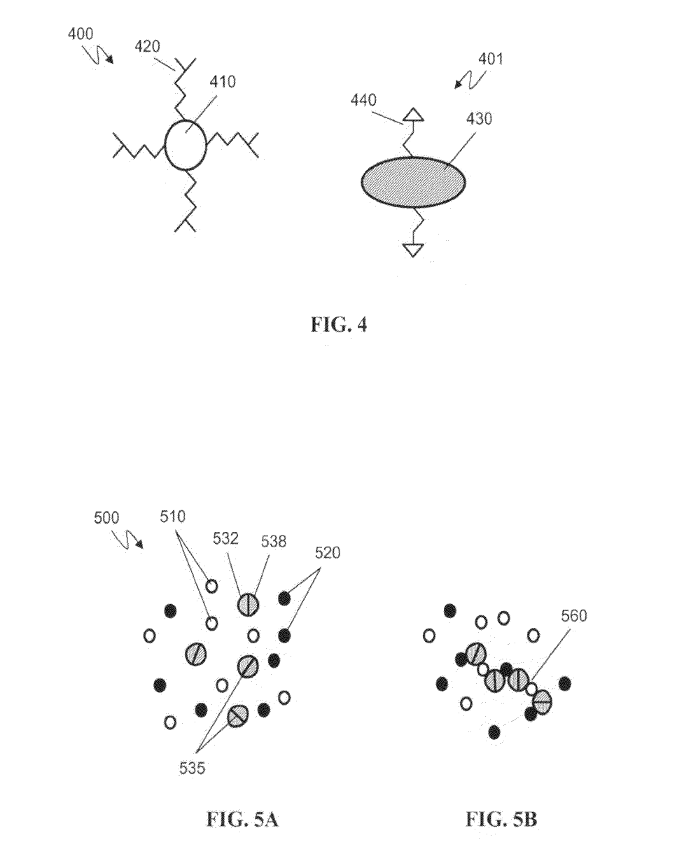

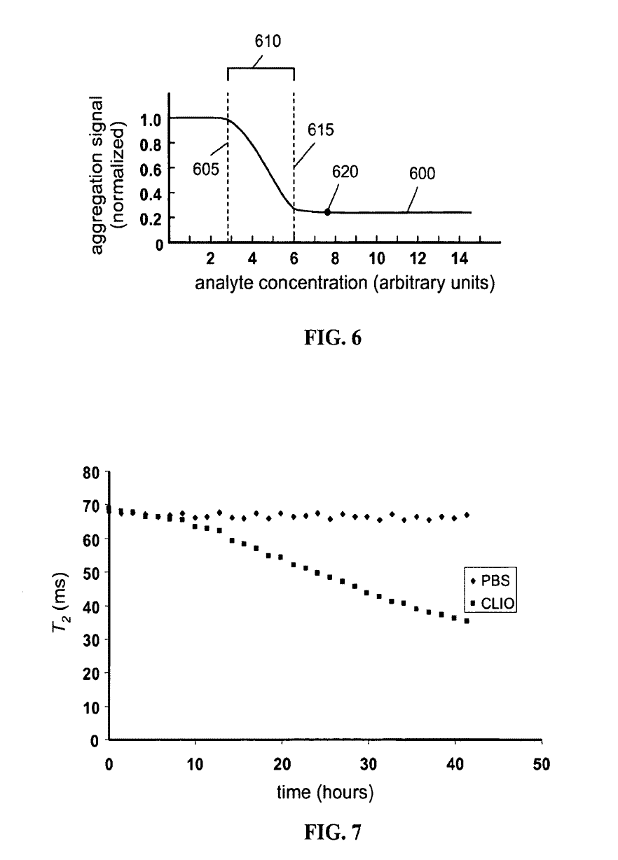

FIGS. 4-5 depict certain embodiments of anti-analytes and analytes. The particular embodiment of FIG. 4 depicts an anti-analyte 400 comprising a chemically-functionalized particle. The particular embodiment of FIGS. 5A-5B depicts an aggregation system in which two types of anti-analytes 510 and 520 are used.

Referring to the embodiment of FIG. 4, a particle 410 may be chemically-functionalized with a ligand 420. It will be appreciated that in various embodiments, 420 may be a receptor. For simplicity, the following uses a ligand 420 for illustrative purposes. Plural ligands 420 may be chemically attached to the surface of a particle 410. The ligands 420 may preferentially bind to receptors 440 located on an analyte 430. Similarly, when using a receptor 420, the receptor may preferentially bind to a ligand 440 located on an analyte 430.

The particle 410 may be micron sized, e.g., having a diameter of any value between about 1 micron and about 250 microns, or may be a nanoparticle, e.g., having a diameter of any value less than about 1 micron. In some embodiments, the particle 410 may be an iron-oxide nanoparticle, a cross-linked iron-oxide nanoparticle, a polymeric nanoparticle, a ceramic nanoparticle, a semi-metal nanoparticle, a semi-conductor nanoparticle, a glass nanoparticle, or a metallic nanoparticle. In some embodiments, the size of the nanoparticle may be between about 10 nanometers and about 100 nanometers, and in some embodiments, between about 100 nanometers and about 200 nanometers. Anti-analytes 130 may comprise particles having similar diameters, e.g., about 50 nm±about 10 nm, about 100 nm±about 20 nm, etc. In some embodiments, anti-analytes may have a narrow distribution of sizes, e.g., less than about 25% of the average particle size. In some embodiments, anti-analytes may have a borad distribution of sizes, e.g., greater than about 25% of the average particle size.

In various embodiments where particles are functionalized with ligands or receptors to form anti-analytes, the ligands or receptors may be selected based upon their ability to target or bind with a particular analyte of interest. Without limitation, the ligand or receptor may comprise antibodies (polyclonal or monoclonal) for the analyte of interest (e.g., a protein biomarker). Conversely, when the analyte is itself an antibody, the ligand may comprise an antigen for that antibody.

Those skilled in the art will recognize alternatives to antibodies. In particular, the present invention also encompasses the use of synthetic anti-analytes that mimic the functions of antibodies. Several approaches to designing and/or identifying antibody mimics have been proposed and demonstrated (e.g., see the reviews by Hsieh-Wilson et al., Acc. Chem. Res. 29:164, 2000 and Peczuh and Hamilton, Chem. Rev. 100:2479, 2000). For example, small molecules that bind protein surfaces in a fashion similar to that of natural proteins have been identified by screening synthetic libraries of small molecules or natural product isolates (e.g., see Gallop et al., J. Med. Chem. 37:1233, 1994; Gordon et al., J. Med. Chem. 37:1385, 1994; DeWitt et al., Proc. Natl. Acad. Sci. U.S.A. 90:6909, 1993; Bunin et al., Proc. Natl. Acad. Sci. U.S.A. 91:4708, 1994; Virgilio and Ellman, J. Am. Chem. Soc. 116:11580, 1994; Wang et al., J. Med. Chem. 38:2995, 1995; and Kick and Ellman, J. Med. Chem. 38:1427, 1995). Similarly, combinatorial approaches have been successfully applied to screen libraries of peptides and polypeptides for their ability to bind a range of proteins (e.g., see Cull et al., Proc. Natl. Acad. Sci. U.S.A. 89:1865, 1992; Mattheakis et al., Proc. Natl. Acad. Sci. U.S.A. 91:9022, 1994; Scott and Smith, Science 249:386, 1990; Devlin et al., Science 249:404, 1990; Corey et al., Gene 128:129, 1993; Bray et al., Tetrahedron Lett. 31:5811, 1990; Fodor et al., Science 251:767, 1991; Houghten et al., Nature 354:84, 1991; Lam et al., Nature 354:82, 1991; Blake and Litzi-Davis, Bioconjugate Chem. 3:510, 1992; Needels et al., Proc. Natl. Acad. Sci. U.S.A. 90:10700, 1993; and Ohlmeyer et al., Proc. Natl. Acad. Sci. U.S.A. 90:10922, 1993). Similar approaches have also been used to study carbohydrate-protein interactions (e.g., see Oldenburg et al., Proc. Natl. Acad. Sci. U.S.A. 89:5393, 1992) and polynucleotide-protein interactions (e.g., see Ellington and Szostak, Nature 346:818, 1990 and Tuerk and Gold, Science 249:505, 1990). These approaches have also been extended to study interactions between proteins and unnatural biopolymers such as oligocarbamates, oligoureas, oligosulfones, etc. (e.g., see Zuckermann et al., J. Am. Chem. Soc. 114:10646, 1992; Simon et al., Proc. Natl. Acad. Sci. U.S.A. 89:9367, 1992; Zuckermann et al., J. Med. Chem. 37:2678, 1994; Burgess et al., Angew. Chem., Int. Ed. Engl. 34:907, 1995; and Cho et al., Science 261:1303, 1993). Yet further, alternative protein scaffolds that are loosely based around the basic fold of antibody molecules have been suggested and may be used in the preparation of inventive anti-analytes (e.g., see Ku and Schultz Proc. Natl. Acad. Sci. U.S.A. 92:6552, 1995).

In some embodiments, more than one type of anti-analyte may be used, as depicted in FIGS. 5A-5B. This may be beneficial when the analyte 535 is not multivalent, e.g., cannot bind to more than one anti-analyte of a particular type at a time, but can bind to more than one type of anti-analyte at a time. For example, an analyte 535 may contain two types of binding sites 532 and 538, which each bind to only one particular anti-analyte type 520 and 510, respectively. In such an aggregation system 500, aggregate products 560 can form due to the presence of the two types of anti-analytes 510 and 520. It will be appreciated, particularly with reference to FIG. 4, that the two types of anti-analytes can comprise substantially identical core particles with different binding affinities, e.g., each anti-analyte 510 and 520 may comprise a core particle 410 with a different ligand 420.

The examples presented below describe a double-anti-analyte aggregation system in which the analyte is human chorionic gonadotrophin (hCG). hCG is not multivalent but has two types of epitopes which bind to a matched pair of monoclonal antibodies, designated as mAb 95 and mAb 97. Each type of antibody can be chemically functionalized onto the surface of nanoparticles, e.g., onto the surface of cross-linked iron-oxide nanoparticles. In certain embodiments, plural antibodies of one type, e.g., the mAb 95 antibody, can be functionalized onto the surface of a nanoparticle to comprise a first type of anti-analyte. Plural antibodies of a second type, e.g., the mAb 97 antibody, can be functionalized onto the surface of another nanoparticle to comprise a second type of anti-analyte. Each type of anti-analyte can bind to their corresponding epitope type on the analyte. Since each anti-analyte type has plural ligands disposed on its surface, they can bind to additional analytes and form a network of bound anti-analytes and analytes as depicted in FIG. 5B.

In various embodiments, the anti-analyte 130 may include a reporter. A reporter is a component which can alter a signal or provide a detectable signal indicative of aggregate formation. In some embodiments, chemical components, e.g., molecules, compounds, proteins, etc., added to the anti-analyte or the analyte may serve as reporters. In certain embodiments, the reporter may be an iron-oxide nanoparticle, a cross-linked iron-oxide nanoparticle, a polymeric nanoparticle, a ceramic nanoparticle, a semi-metal nanoparticle, a semi-conductor nanoparticle, a glass nanoparticle, or a metallic nanoparticle. Any means of detecting a signal provided by, or altered by, reporters may be used. For example in some embodiments, optical fluorescence detection may be used to detect a signal provided by, or altered by, reporters. An optical beam of radiation may be used to probe the matrix and excite fluorescence in reporters. The fluorescence may be detected by sensitive optical detectors, e.g., photomultipliers. In some embodiments, nuclear magnetic resonance (NMR) may be used to detect a signal provided by, or altered by, reporters. The matrix may be probed with magnetic signals using techniques and methods of NMR, and reporters may alter the detected NMR signal. In various embodiments, a signal from reports has a first characteristic when anti-analytes and analytes are not aggregated, and has a second characteristic when an amount of anti-analytes and analytes are aggregated.

In some embodiments, the anti-analyte 130 itself may alter a signal derived from the analyte 140. For example, the analyte may provide a detectable NMR or fluorescent signal, and the anti-analyte, when bound to the analyte, may alter the frequency, phase or amplitude characteristics of the NMR or fluorescent signal.

In various embodiments, the anti-analytes 130 are mobile in the matrix 120. As used herein, the term “mobile” means that that the anti-analytes are able to move within at least a portion of the matrix. For example, the anti-analytes may move and diffuse through pores within the matrix 120. In various embodiments, the mean size of the anti-analytes is smaller than the mean intrinsic pore size within the matrix. In some embodiments, the mean size of the anti-analytes may be larger than the mean pore size within the matrix, but smaller than the mean pore size within the matrix when the matrix is subjected to a liquid which causes a swelling of the matrix.

In some embodiments, the anti-analyte may comprise aggregates which are substantially immobile within the matrix. Upon introduction of analytes, the anti-analyte aggregates may break apart, dissipate and disperse throughout the matrix. In such an embodiment, the process can be constitute reverse aggregation.

### II. E. Analyte

In various embodiments, the analyte comprises a compound, molecule, protein, nucleotide, antigen, antibody, virus, bacteria, nucleic acid, lipid, carbohydrate, ligand or any chemical or biological marker or species suspected to be present in a sample or specimen. In some embodiments, the analytes may be attached to particles or cells. In other embodiments, analytes may have receptors or ligands disposed on their surface. The analyte may have multiple binding sites of one type, or may have multiple types of binding sites.

Referring to FIG. 4, in some embodiments an analyte 401 may have disposed on its surface naturally occurring receptors or ligands 440 which bind with mating ligands or receptors on an anti-analyte. In various embodiments, the receptors or ligands 440 may be chemically attached to the analyte in a human-engineered process, or may naturally comprise at least a portion of the analyte structure. In certain embodiments, an analyte 430 may have receptors or ligands 440 chemically functionalized onto its surface.

Referring again to FIG. 4, in some embodiments, the analytes may be the receptors or ligands 440 themselves, which are bound to a core 430. As an example, the analytes may be antigens disposed on the surface of biological cells, such as antigens disposed on the surface of red blood cells in a condition indicative of immune mediated haemolytic anemia.

As described in the examples below, human chorionic gonadotrophin (hCG) is an analyte having multiple types of binding sites and which has an alpha subunit (hCG-α) and a beta subunit (hCG-β). Each subunit of this peptide hormone preferentially binds to only one particular monoclonal antibody. Correct detection of this human biomarker can be important, since it can indicate certain oncological malignancies such as testicular and ovarian cancer, and also may indicate pregnancy.

In various embodiments, the analytes 140 are provided in a solution 150, and the solution is brought into contact with the matrix 120 containing anti-analytes 130. The solution suspected to contain analytes may be undiluted, e.g., as drawn from a subject, or may be diluted in a liquid, e.g., dilution in distilled water, saline solution, alcohol, phosphate buffered saline (PBS). The dilution of a sample suspected to contain an analyte may be to a known and predetermine dilution amount, e.g., any dilution value between about 0% and about 100% where a 100% value indicates a non-diluted sample, and a 10% value indicates a mixture, by weight or volume, of 10% sample suspected to contain an analyte and 90% dilution liquid. It will also be appreciated that the solution 150 may be processed, e.g., by purification, chromatography, etc., or may be unprocessed.

Any of a wide variety of analytes may be used in the embodiments described herein, as will be appreciated by one skilled in the art of agglutination assays or immunoassays. It will further be appreciated by one skilled in the art that the embodiments herein will be particularly useful when analytes or their agglutination products are unstable in solution, e.g., precipitate out of solution.

### II. F. Mobility of Constituents within the Matrix

In various embodiments, anti-analytes 130 and analytes 140 are mobile within the matrix, as indicated in the depictions of FIGS. 1B-1D. Both anti-analytes and analytes may diffuse throughout the porous or semi-porous matrix 120. As they encounter each other, aggregates 160 can form. In some embodiments, aggregates larger than a certain size are substantially immobile within the material. In various embodiments, the substantially immobile aggregates become lodged within the matrix, and may be substantially evenly dispersed throughout at least a portion of the volume defined by the matrix 120. In various embodiments, the lodged aggregates remain substantially stationary within the matrix and provide, alter or affect at least one signal to indicate formation of aggregates.

As time elapses after the addition of a solution 150, the analytes 140 diffuse into the matrix 120 as depicted in FIGS. 1C-1D. In various embodiments, the analytes 140 and anti-analytes 130 move within the matrix 120 and form aggregation products 160a, 160b, 160c as depicted in FIG. 1D. In some embodiments, small aggregates, e.g., aggregate 160a, may be mobile within the matrix 120. In various embodiments, large aggregates, e.g., aggregates 160b and 160c, become substantially immobile within the matrix and remain substantially lodged at their location. The lodged aggregates can remain suspended in the matrix 120 for durations of time exceeding about 30 minutes, about 1 hour, about 2 hours, about 5 hours, about 10 hours, about 20 hours, about 40 hours and yet about 80 hours. In various embodiments, the size of the lodged aggregates, e.g., aggregates 160b and 160c, may continue to grow if supplied with analytes 140 and anti-analytes 130 from within the matrix 120. In some embodiments, the growth of aggregates may be limited as anti-analytes 130 become depleted within the matrix. When multiple aggregates form, regions in the immediate vicinity of each aggregate may become depleted of anti-analytes 130 and analytes 140, which in turn can control or limit further growth of the lodged aggregates.

In various embodiments, the lodged aggregates have a size greater than the mean intrinsic pore size within the matrix 120. In some embodiments, the size of the lodged aggregates is about 10% larger than the mean pore size within the matrix, about 20% larger than the mean pore size within the matrix, about 30% larger than the mean pore size within the matrix, about 50% larger than the mean pore size within the matrix, about 75% larger than the mean pore size within the matrix, and about 100% larger than the mean pore size within the matrix.

In various embodiments, mobility of anti-analytes 130 and analytes 140 within the matrix 120 can be affected by selection of the matrix material. In some embodiments, the matrix 120 can be selected based on its mean intrinsic pore size and to limit the maximum size of mobile aggregates within the matrix, e.g., to limit the size of lodged aggregates within the matrix. As an example, a matrix 120 having a mean intrinsic pore size of about 200 nm may be selected when it is desired that aggregates larger than about 200 nm should become substantially immobile within the matrix. As an example, a matrix 120 having a mean intrinsic pore size of about 400 nm may be selected when it is desired that aggregates larger than about 400 nm should become substantially immobile within the matrix. As an example, a matrix 120 having a mean intrinsic pore size of about 800 nm may be selected when it is desired that aggregates larger than about 800 nm should become substantially immobile within the matrix. As an example, a matrix 120 having a mean intrinsic pore size of about 2 microns may be selected when it is desired that aggregates larger than about 2 microns should become substantially immobile within the matrix. As an example, a matrix 120 having a mean intrinsic pore size of about 4 microns may be selected when it is desired that aggregates larger than about 4 microns should become substantially immobile within the matrix. As an example, a matrix 120 having a mean intrinsic pore size of about 8 microns may be selected when it is desired that aggregates larger than about 8 microns should become substantially immobile within the matrix. As yet another example, a matrix 120 having a mean intrinsic pore size of about 16 microns may be selected when it is desired that aggregates larger than about 16 microns should become substantially immobile within the matrix.

In various embodiments, mobility of anti-analytes, analytes or aggregates within the matrix may be controllably and temporarily altered or enhanced. For example, the matrix containing the aggregation constituents may be heated, or subjected to ultrasonic agitation, or subjected to centrifugal force, or subjected to a pressurized environment, or subjected to applied electric or magnetic fields, or subjected to shaking. Any of these process steps may increase mobility of anti-analytes, analytes, and/or aggregates within the matrix.

### II. G. Measurement Volume

As depicted in FIG. 1D a measurement volume 180 is provided within the matrix 120. The measurement volume may comprise at least a portion of the matrix material 120, which can be probed to provide a signal representative of aggregate formation. In certain embodiments, the measurement volume 180 comprises a thin slab-like section of the matrix, e.g., a slab between about 100 microns and about 300 microns thick, or between about 300 microns and about 600 microns thick, or between about 600 microns and about 1 millimeter thick. The slab-like section may extend across the entire lateral dimension of the vessel 110, or may extend across a portion of the lateral dimension of the vessel. The slab-like section may be located near the top of the matrix, or at any depth within the matrix. In some embodiments, the measurement volume 180 may be in the shape of a cube, rectangle, sphere, or oblate sphere located within the matrix 120. In some embodiments, the measurement volume may comprise substantially the entire volume occupied by the matrix 120.

### II. H. Alterations to Improve Operability of Matrix-Stabilized Aggregation

In various embodiments, several parameters characterizing the matrix and anti-analytes can be selected or altered to improve operability of matrix-stabilized aggregation assays. Such selections and/or alterations may be beneficial when test solutions have widely varying analyte concentrations. The parameters that can be selected or altered include, but are not limited to, concentration of anti-analytes 130, temperature of the matrix 120, and mean intrinsic pore size within the matrix 120. As an example, when the test solution 150 has a high concentration of analytes, a reduction in the mean pore size of the material 120 may ameliorate uncontrolled aggregation. Accordingly, a matrix material may be selected with a smaller pore size than that used for an aggregation assay in which uncontrolled aggregation was observed. In general, uncontrolled aggregation can lead to an undesirable signal saturation effect. A signal normally representative of an amount of aggregation would become saturated when further increases in analyte concentration would produce substantially no change in the detected signal from the measurement volume 180. A reduction in mean pore size can limit the size of aggregates formed in the material, and may ameliorate uncontrolled aggregation and saturation of a signal representative of an amount of aggregation. In some embodiments, a reduced concentration of anti-analytes 130 within the matrix 120 may also limit the extent of aggregation when the concentration of analytes is high. When anti-analytes are sparse, they may become depleted from the matrix more quickly when aggregates form, as compared to an embodiment where the anti-analytes are present in high concentration. Their rapid depletion may limit the extent of aggregate formation and may ameliorate uncontrolled aggregation and saturation of a signal representative of an amount of aggregation.

For embodiments where the concentration of analytes 140 in solution 150 may be low, the mean pore size and/or the concentration of anti-analytes 130 can be increased. The increase in pore size and/or anti-analyte concentration can permit larger aggregates to form within the matrix 120. The larger aggregates may produce a detectable signal representative of aggregate formation, whereas multiple smaller aggregates may not produce a detectable signal as might be the case when smaller pore sizes and/or lower anti-analyte concentrations are used.

In certain embodiments, the matrix 120 with anti-analytes 130 may be provided in a plurality of samples having different mean intrinsic pore sizes and/or anti-analyte concentrations. For example, the matrix 120 with anti-analytes 130 may be provided in a variety of samples in a multi-well plate. For example, each sample within a row of samples in the plate may provide a different mean intrinsic pore size, and all samples within the row span a range of mean intrinsic pore sizes. Values of mean intrinsic pore size from row to row may be substantially the same. Each sample within a column of samples in the plate may provide a different anti-analyte concentration, and all samples within the column span a range of anti-analyte concentrations. Values of anti-analyte concentration from column to column may be substantially the same.

In some embodiments, the temperature of the matrix 120 may be controlled during aggregate formation. For example in some embodiments, the temperature of the matrix may be elevated to increase mobility of anti-analytes and analytes within the matrix. Increased mobility may encourage formation of aggregates, and be useful for low analyte concentrations. In some embodiments, the temperature of the matrix may be reduced to decrease mobility of anti-analytes and analytes within the matrix. Decreased mobility may inhibit formation of aggregates, and be useful for high analyte concentrations.

Accordingly, alterations and/or selections of aggregation system parameters can be implemented to improve operation of aggregation assays. The alterations or selections may avoid uncontrollable aggregation, prevent saturation of signals representative of aggregate formation, promote aggregation, or inhibit aggregation. The alterations can include, in any combination and without being limited to, altering or selecting an anti-analyte concentration, altering, selecting or controlling the temperature of the matrix, and selecting a matrix with a desired mean intrinsic pore size.

### III. Methods of Matrix-Stabilized Aggregation

### III. A. Overview of Matrix-Stabilized Aggregation Methods

An embodiment of a method 200 for quantitatively determining a concentration of an analyte in a test solution is depicted in the flow diagram of FIG. 2. In some embodiments, the matrix-stabilized aggregation method 200 comprises steps of providing (step 210) a matrix comprising a substance having a viscosity greater than about 1.5 centipoise and anti-analytes dispersed within the substance, contacting (step 220) a test solution containing a concentration of analytes to the matrix so that the analytes permeate through at least a portion of the matrix, and detecting (step 230) a signal representative of an amount of aggregates that form within a volume of the matrix. In various embodiments, the anti-analytes and analytes are mobile within the matrix, and aggregates larger than a certain size are substantially immobile within the matrix. For example, a certain size of aggregates which may become substantially immobile within the matrix may be a size between about 200 nm and about 400 nm, a size between about 400 nm and about 800 nm, a size between about 800 nm and about 2 microns, a size between about 2 microns and about 4 microns, a size between about 4 microns and about 8 microns, and yet in some embodiments, a size between about 8 microns and about 16 microns.

The step of providing the matrix (step 210) can include providing the matrix in a vessel. The vessel may contain an amount of matrix with anti-analytes dispersed within the matrix, and the vessel may be coverable. In some embodiments, the matrix may be provided in a vessel adapted for placement in vivo. The step of providing may include placing a vessel containing an amount of the matrix 120 with anti-analytes 130 in vivo. In some embodiments, the matrix with anti-analytes dispersed therein may be provided in plural vessels, e.g., plural vessels disposed in an array such as found in 24-, 48-, 96-, and 384-well plates. In some embodiments, plural discrete amounts of matrix with anti-analytes dispersed therein may be provided in an array of dots or spots, e.g., an array of spots on a microtitre plate. matrix with anti-analytes dispersed within may be obtained in various packaged arrangements from a supplier, or prepared in accordance with techniques described herein, and thereafter provided for a subsequent aggregation test or assay.

In some embodiments, the step of providing the matrix (step 210) includes preparing an amount of matrix with anti-analytes dispersed within. For example, the matrix material and anti-analytes may be provided separately or stored separately, and mixed by a user prior to carrying out an agglutination test. The mixing may involve rendering the matrix into a molten or flowable state before introducing anti-analytes. The mixture may be allowed to set, e.g., return to a solid, substantially solid, semi-solid, or gel state, prior to use in an agglutination test.

The step of contacting a test solution to the matrix (step 220) can include introducing an amount of test solution containing a concentration of analytes into physical contact with the matrix. The test solution may be added into a vessel containing an amount of the matrix, as depicted in FIG. 1B. In some embodiments, the test solution may be added to an amount of matrix deposited on a substrate, as depicted in FIGS. 1E-1F. In various embodiments, the amount of matrix, the concentration of anti-analytes within the matrix, and the amount of test solution may be measured and/or recorded. In some embodiments, the step of contacting a test solution to the matrix (step 220) may further include increasing or decreasing the mobility of analytes and/or anti-analytes within the matrix via any of the methods described herein. For example, the matrix containing the analytes and anti-analytes may be heated, or subjected to ultrasonic agitation, or subjected to centrifugal force, or subjected to a pressurized environment, or subjected to shaking. In some embodiments, the step of contacting can further include providing a period of time for continued permeation of analytes through at least a portion of the matrix. In various embodiments, the period of time for continued permeation is between about 1 minute and about 5 minutes, between about 5 minutes and about 10 minutes, between about 10 minutes and about 20 minutes, between about 20 minutes and about 40 minutes, between about 40 minutes and about 1 hour, between about 1 hour and about 2 hours, between about 2 hours and about 4 hours, between about 4 hours and about 8 hours, and yet between about 8 hours and about 16 hours.

The step of detecting a signal representative of an amount of aggregates (step 230) can comprise subjecting at least a portion of the matrix to a measurement. The measurement may include probing at least a portion of the matrix with optical, electronic, or magnetic fields, and monitoring the probing field for a change, or monitoring for a response, indicative of aggregate formation within the matrix. As an example, in some embodiments, the probing field may be an optical beam of radiation incident upon at least a portion of the matrix. A response to the probing optical beam may be fluorescence from the probed portion of the matrix. The amount of fluorescence detected may be increased or decreased in the presence of aggregates, as compared to a similar matrix having no aggregates, and be representative an amount of aggregates that form within a volume of the matrix. As an additional example, in some embodiments, at least a portion of the matrix may be probed with magnetic fields produced by nuclear magnetic resonance instruments, e.g., by a Minispec homogeneous field relaxometer available from Bruker Optics, Billerica, Mass., or an ex situ magnetic resonance sensor available from ACT, Aachen, Germany. These instruments may be adapted to measure longitudinal T₁ or transverse T₂ relaxation times for samples within the probing field. Changes in the measured times may be representative an amount of aggregates that form within a volume of the matrix. In some embodiments where an amount of matrix is placed in vivo, a medical magnetic resonance imaging (MRI) system may be used to detect in vivo a signal representative of aggregate formation within the matrix.

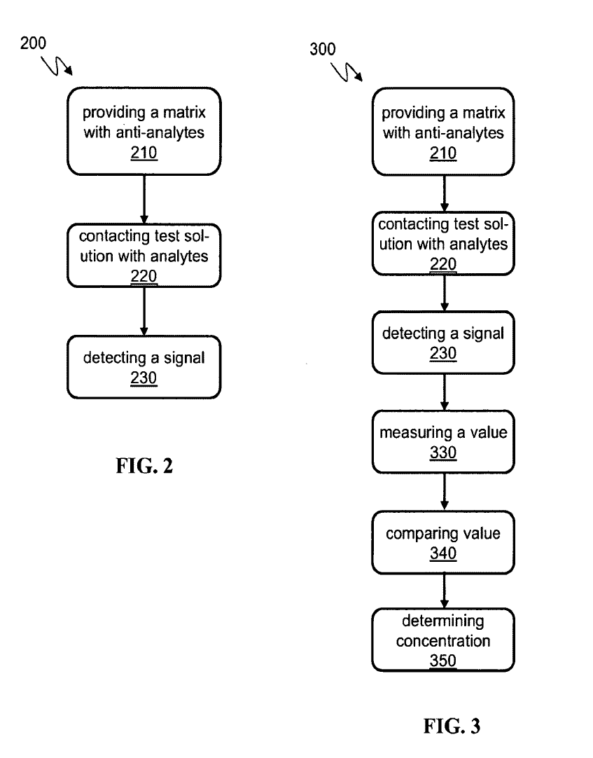

The flow chart of FIG. 3 depicts an additional embodiment of a method 300 for matrix-stabilized aggregation-based assays. The method 300 includes steps of measuring (step 330) a value of the detected signal, comparing (step 340) the measured value with calibration standards, and determining (step 350) an analyte concentration based upon the comparison in step 340. The step of measuring a value (step 330) can include recording or noting a value or signal level of the detected signal representative of aggregate formation, e.g., an amount of fluorescence, an amount of optical scattering or absorption, an amount of X-ray absorption, or longitudinal T₁ or transverse T₂ relaxation times. The measured value may be recorded electronically and stored in electronic or magnetic memory for later retrieval. The measured value may be compared (step 340) to calibration standards, e.g., a tabulation of similar measured values determined previously in controlled trials. In various embodiments, the prior tabulated values are obtained from trials carried out with known concentrations of analytes, and provide a similarly measured value associated with a known analyte concentration. As a result of the comparison (step 340), the step of determining a concentration of the analyte (step 350) can return a quantitative value of the concentration of the analyte in the test solution.

### III. B. Accelerating Aggregate Formation

The inventive methods 200 and 300 can further include steps which accelerate diffusion and aggregate formation in certain embodiments. For embodiments where the anti-analyte 130 or analyte 140 may comprise a magnetic component, continuous or alternating magnetic fields may be applied to a region containing the matrix 120 to increase the mobility of the anti-analytes or analytes within the matrix. For embodiments where the anti-analyte or analyte may carry a net electrical charge, continuous or alternating electric fields may be applied to a region containing the matrix to increase either or both constituents' mobility within the matrix. Other mobility-inducing techniques may also be used. For example, a vessel 110 containing the matrix may be covered and pressure applied to the region 115 within the vessel. A cover may contain an opening through which a pressurized gas can be introduced into the vessel to elevate its internal pressure above about 1 atmosphere (atm), above about 2 atm, above about 4 atm, and in some embodiments above about 8 atm. In some embodiments, the vessel may be placed in a mechanical apparatus which can apply centrifugal force to the vessel, or shake the vessel. In yet other embodiments, ultrasonic vibrations can be coupled to the vessel and/or matrix to induce ultrasonic agitation to the aggregation system.

In some embodiments, the anti-analyte and/or analyte may be modified by chemical engineering to enable means for accelerating aggregate formation as described above. As an example and referring to FIG. 4, a ligand 420 and/or receptor 440 can be engineered to comprise a magnetic component or to carry a net electrical charge. Such a ligand or receptor may be attached to the anti-analytes or analytes, so that continuous or alternating magnetic or electric fields may impart force to the anti-analytes or analytes.

In various embodiments, when methods 200 or 300 includes a step of accelerating diffusion or aggregation within the vessel, the period of time associated with allowing diffusion or permeation of analytes into the matrix may be reduced.

### III. C. Detecting Aggregate Formation

After aggregate products 160 form in the matrix 120, measurements can be made to detect a signal representative of an amount of aggregation within the matrix. In various aspects, the measurement is made within a measurement volume 180 of the matrix. In some embodiments, the measurement volume 180 may comprise a portion of the volume occupied by the matrix, and in some embodiments, the measurement volume 180 may comprise substantially all of the volume occupied by the matrix. Various types of measurements can be carried out and may include, without limitation, any of the following techniques: nuclear-magnetic-resonance (NMR) imaging, nuclear-magnetic-resonance spectroscopy, nuclear magnetic relaxometry, optical scattering, optical spectroscopy, optical imaging, optical fluorescence, infrared imaging, infrared spectroscopy, infrared scattering, and X-ray imaging. Each of these techniques may provide a signal representative of an amount of aggregation which has occurred within the measurement volume 180. Signals may be derived from the aggregates, the anti-analytes, the analytes, or reporter components attached to the analytes or anti-analytes. In some embodiments, protons (e.g., in water), isotopes, elements or molecules uniformly distributed throughout the measurement region may serve as reporters providing a signal indicative of aggregate formation. In some embodiments, multiple measurement techniques may be used, and different signals representative of an amount of aggregation may be obtained.

As an example of a measurement technique which can detect a signal indicative of aggregate formation, nuclear magnetic resonance (NMR) may be used to measure a spin-spin, or transverse, relaxation time T₂ within a measurement volume 180. The T₂ time may be a characteristic NMR signal derived from the anti-analyte 130, or the analyte 140, or a reporter or molecule attached to the anti-analyte or analyte. Referring to FIGS. 4A-4B, it will be appreciated by one skilled in the art that any of the aggregate components, e.g., the particle 410, the ligand 420, the analyte 430, or the receptor 440, can participate in providing a signal indicative of aggregate formation. In some embodiments, the T₂ time may be derived from protons (e.g., in water) within the measurement region 180.

Returning now to the NMR example, as aggregates form within the region 180 the measured T₂ time associated with a particular constituent can be altered due to changes in the local density of material, the local magnetic susceptibility or both. For example, a clustering of anti-analytes comprising iron-oxide nanoparticles can change both the local density and local magnetic susceptibility. The amount of change in the measured T₂ time, or the final measured value of T₂, after the addition of a solution 150 suspected to contain an analyte, can be representative of an amount of aggregation of analytes 140 and anti-analytes 130 within the measurement region 180. It will be appreciated that NMR techniques can be used to measure spin-lattice, or longitudinal relaxation times T₁, which may also provide a signal indicative of an amount of aggregation.

As another example of a measurement technique providing a signal representative of an amount of aggregate formation, optical fluorescence methods may be employed to detect a signal within a measurement volume 180. In this example, fluorescent molecules may be chemically attached to any of the following aggregation constituents: the anti-analyte 130, the analyte 140, a ligand bound to the anti-analyte or analyte, or a receptor bound to the anti-analyte or analyte. In some embodiments, the ligand or receptor may fluoresce. The fluorescent molecule may emit radiation at a particular wavelength when excited with radiation at a different, typically shorter, wavelength. For example, a fluorescent molecule may emit red radiation, when excited or pumped with green radiation. In certain embodiments, the fluorescent molecule may normally emit radiation when excited, but become quenched or suppressed when bound in an aggregate network. Accordingly, a decrease in fluorescent radiation from a measurement volume 180 within the matrix can indicate the formation of aggregates, and the amount of decrease can be representative of a concentration of analytes. It will be appreciated that some embodiments may be implemented for which normally suppressed fluorescent molecules become fluorescent when aggregates form, e.g., a quenched fluorescent molecule in a ligand or receptor may become non-quenched when the ligand or receptor binds to a target molecule.

In various embodiments, the signals from a matrix-stabilized aggregation system which are representative of an amount of aggregate formation attain a substantially stable value that persists for periods of time longer than about 30 minutes, than about 1 hour, than about 2 hours, than about 4 hours, than about 8 hours, than about 16 hours, and in some embodiments longer than about 32 hours. In various embodiments, the signal attains a substantially stable value within about 10 hours, about 5 hours, about 2 hours, about 1 hour, about 30 minutes, about 15 minutes, about 10 minutes, about 5 minutes, and yet about 2 minutes after allowing diffusion of the analytes into the matrix. In some embodiments, the signal indicative of aggregate formation may pass through a transition period during which the signal changes from an initial value, prior to the diffusion of analytes into the matrix, to a substantially stable value after diffusion of analytes into the matrix. The transition period from an initial value to a substantially stable value may last for less than about 5 hours, less than about 2 hours, less than about 1 hour, less than about 30 minutes, less than about 15 minutes, less than about 10 minutes, less than about 5 minutes, less than about 2 minutes, and yet less than about 1 minute in various embodiments. After attaining a stable value, the value of a signal indicative of an amount of aggregate formation may vary during any one of these time periods by less than about ±25%, ±20%, ±15%, ±10%, and yet less than about ±5% in some embodiments.

### III. D. Determining and Using Calibration Standards

In certain embodiments, the calibration standards associated with the step of comparing (step 340) may be determined by providing a matrix which is substantially similar to the matrix used for aggregation tests. For example, the matrix may comprise a substance having a viscosity greater than about 1.5 centipoise and anti-analytes dispersed within the substance and mobile within the substance. The calibration standards may further be determined by contacting a test solution containing a known concentration of analytes to the matrix so that the analytes permeate through at least a portion of the matrix, and detecting a value of a signal representative of an amount of aggregates that form within a volume of the matrix. In various embodiments, the aggregates form from the anti-analytes dispersed within the matrix and the analytes. The calibration standards may further be determined by recording the detected value and associating the value with the known concentration of analytes, and repeating the steps of providing, contacting, detecting and recording for different known concentrations of the analytes in solution. The measured values of the detected signals, e.g., changes in T₁ or T₂ times, fluorescent radiation, optical absorption, etc., for each calibration run can be tabulated to provide a set of calibration standards. In this manner, calibration values can be tabulated for different known concentration of analytes. Accordingly, a measured change in signal value for a test solution with an unknown concentration of analytes can be compared with the tabulated calibration values to determine quantitatively a concentration of the analytes in the test solution.

In various embodiments, calibration measurements can be carried out prior to aggregation tests, and results from the calibration measurements can be used to determining quantitatively a concentration of analytes in tested solutions. The calibration standards can span a wide range of analyte concentrations and system parameters. In some embodiments, a list or data bank of calibration values and associated analyte concentrations may be provided or supplied the matrix.

### III. E. Dynamic Range

In various embodiments, dynamic ranges can be determined for various analyte concentrations. As used herein, a dynamic range is a range of detected signal values over which the detected signal value represents, substantially accurately, a unique concentration of analytes. FIG. 6 is an illustrational graph depicting a dynamic range 610 of a hypothetical aggregation signal 600, which is plotted as a function of analyte concentration. In practice, data necessary to generate the aggregation signal 600 for a particular analyte and aggregation system may be obtained from calibration trials. As can be seen from the graph, within the dynamic range 610 each value of the analyte concentration corresponds to a unique value of the detected aggregation signal, and vice versa. In some embodiments, the dynamic range 610 may be substantially linear. In some embodiments, the dynamic range 610 may be non-linear. In some embodiments, the aggregation signal may decrease with increasing analyte concentration, and in other embodiments, the aggregation signal may increase with increasing analyte concentration. When a detected aggregation signal value falls within a dynamic range, then the measured signal value, e.g., the value measured in step 330, can provide quantitatively, and substantially accurately, a value of analyte concentration.

Dynamic ranges for various embodiments of aggregation systems can be determined by carrying out calibration trials wherein solutions containing known concentrations of analytes are introduced to particular matrix materials 120 with known concentrations of anti-analytes 130 dispersed therein. In some embodiments, each particular matrix material may have a known mean intrinsic pore size. Each of several parameters, e.g., analyte concentration, type of analyte, analyte solution, matrix material, mean pore size within the matrix, anti-analyte concentration, anti-analyte size, and temperature can be varied in turn to determine the dynamic ranges for various embodiments of the inventive aggregation system.

As an example for an embodiment of an aggregation system having a first matrix material with a first mean pore size and a first anti-analyte concentration, a lower bound 605 of a first dynamic range DR₁ would be established as the point below which a decrease in analyte concentration produces substantially no detectable change in a signal indicative of an amount of aggregate formation within a measurement volume 180. Additionally, an upper bound 615 of the first dynamic range would be established as the point above which an increase in analyte concentration produces substantially no detectable change in a signal indicative of an amount of aggregate formation. A second dynamic range DR₂ could be determined in a similar manner wherein a second mean pore size is used with the first anti-analyte concentration. A third dynamic range DR₃ could be determined wherein the first mean pore size is used with a second anti-analyte concentration. Accordingly, numerous dynamic ranges can be determined and recorded for a variety of embodiments having different mean pore sizes, different anti-analyte concentrations, different matrix materials, and different analyte concentrations. Data recorded from trials to determine dynamic ranges may be stored and subsequently consulted when performing aggregation tests with unknown concentrations of analytes. For example, the dynamic range data may be used in the step of comparing (step 340) a measured value with calibration standards.

As an additional approach to determining dynamic ranges, Monte Carlo simulations could be developed and carried out to simulate aggregation processes within various matrix materials 120. Adjustable parameters for the simulation could include mean pore size, anti-analyte concentration, anti-analyte size, mobility of the anti-analyte within the matrix, temperature, analyte concentration, analyte size, and mobility of the analyte within the matrix. Results from the Monte Carlo simulations could be used to guide experimental determination of dynamic ranges, or experimental tests may validate results from the Monte Carlo simulations.

Once the dynamic range is determined for a particular matrix 120 with anti-analytes 130, e.g. a matrix of a particular material with a certain mean intrinsic pore size and anti-analyte concentration, information about the dynamic range may be provided with the matrix. Information about the dynamic range can aid in the selection of a particular matrix with anti-analytes for anticipated analyte concentrations.

It will be appreciated by one skilled in the art that the matrix 120, which may be characterized by its mean intrinsic pore size and/or dynamic range, will generally be selected in relation to one or more of the following quantities: mean size of the anti-analytes 130, mean size of the analytes 140, desired mean size of aggregate products 160, dynamic range, and anticipated concentration of analytes. It will further be appreciated by one skilled in the art that the concentration of anti-analytes 130 in the matrix 120 will be selected based upon one or more of the following quantities: anticipated concentration of analytes 140, and mean pore size within the matrix 120. In various embodiments, the mean pore size and concentration of anti-analytes will be selected to provide a dynamic range for a test solution suspected to have an analyte present within an anticipated range of concentrations.

In cases where an aggregation test produces a signal which falls outside a dynamic range, the test may be repeated using an altered analyte concentration. As an example and referring to FIG. 6, if an aggregation test produces a signal value 620 which is greater than the upper bound 615 of the dynamic range, the analyte concentration may be diluted and the test repeated. If an aggregation test produces a signal which falls below the lower bound 605 of the dynamic range, then the analyte concentration may be condensed, e.g., by evaporation of liquid or centrifugation and removal of supernatant, and the test repeated. Once a detected signal value falls within the dynamic range 610, the concentration of an analyte may be determined from calibration standards and consideration of any dilution or condensing undertaken to place the signal within the dynamic range 610.

In various embodiments, matrix-stabilized aggregation systems provide dynamic ranges which are stable for periods of time exceeding about 1 hour. A stable dynamic range is one in which signal values over a range of analyte concentrations do not significantly change with time. In some embodiments, the dynamic range for a particular aggregation system remains substantially unchanged for a period of time exceeding about 2 hours, about 4 hours, about 8 hours, about 16 hours, about 24 hours, about 2 days, about 5 days, and yet about 14 days.

### EXAMPLES

### Example 1

### Mobility of Anti-Analytes within the Matrix

In various embodiments, the anti-analyte is mobile within the matrix 120. FIG. 7 is a plot of experimental data which demonstrates mobility of anti-analytes 130 within a stabilizing matrix 120. For this experiment, an agarose gel was prepared to form the matrix 120. A mixture of 1% agarose gel in deionized water was heated in a microwave oven to form a molten substance. An equal volume of water was added and mixed into the molten substance to produce a 0.5% agarose solution. While still hot, the solution was deposited into vessels, partially filling each vessel, and left for a period of time to set, e.g., cool and solidify. After solidification of the agarose matrix, two trials were carried out. In the first trial, a phosphate buffered solution (PBS) was added to a vessel containing an amount of the matrix. Nuclear magnetic resonance measurements of the transverse relaxation time T₂ of protons (H⁺) within a measurement region within the matrix was carried out at successive time intervals over a period of about 40 hours. The measurement region comprised a slab of the matrix approximately 250-micron thick extending across the vessel, and located at about one-half the height of the matrix. In the second trial, a phosphate buffered solution containing a concentration of about 16 micrograms per milliliter (μg/ml) Fe of cross-linked iron-oxide (CLIO) nanoparticles was added to a vessel. The solution containing the CLIO nanoparticles was contacted to the top of the matrix. The CLIO nanoparticles were prepared as described in Example 2 below. Similar magnetic resonance measurements were made for both the first and second trials over the same period of time.

Results of the mobility measurements (squares) and control measurements (diamonds) are plotted in FIG. 7. The control measurement shows a substantially constant value of the proton transverse relaxation time T₂ (about 68 ms) over a period of about 40 hours. The mobility measurements show a continuously decreasing relaxation time. The decrease in time is attributed to the diffusion of the CLIO nanoparticles from the solution contacted to the top of the matrix into the gel matrix. The reduction in T₂ time can be attributed to the effect of the CLIO nanoparticles on precessing magnetic moments of the protons. As time progresses, the concentration of the nanoparticles increases within the measurement region of the matrix. The results of FIG. 7 indicate mobility of an anti-analyte with a substantially solid matrix.

### Example 2

### Preparation of Functionalized Cross-Linked Iron-Oxide Nanoparticles

In various embodiments, cross-linked iron-oxide (CLIO) nanoparticles are chemically functionalized with ligands which preferentially bind to an analyte. This example describes methods used for the conjugation of particular types of ligands to CLIO nanoparticles. In one experiment, mouse immunoglobulin G (IgG), an antibody to protein A, was conjugated to CLIO nanoparticles. In another experiment, monoclonal antibodies to human chorionic gonadotrophin (hCG) were conjugated to CLIO nanoparticles. The functionalized nanoparticles served as anti-analytes in subsequent experiments.

Magnetic iron oxide nanoparticles with amine terminated dextran shell (CLIO-NH₂) were produced as described in the work of J. L. Tung, et al., “High-efficiency intracellular magnetic labeling with novel superparamagnetic-Tat peptide conjugates,” Bioconjug Chem 10, (1999) pp. 186-191, which is incorporated by reference in its entirety. CLIO-NH₂ was treated with sulfo-succinimidyl-4-(N maleimidomethyl)cyclohexane-1-carboxylate (Sulfo-SMCC available from Pierce, Rockford, Ill.) to create a maleimide functional group. Antibodies (e.g., mouse IgG available from Sigma; and monoclonal antibody (mAb) to beta-hCG available from Scripps Laboratories, San Diego, Calif.) were activated with N-succinimidyl-S-acetylthioacetate (SATA available from Pierce) to generate a blocked sulfanyl group which was de-protected with hydroxylamine. The CLIO-SMCC was incubated with the prepared antibody solutions for 4 to 8 hours at 4° C. The reaction was quenched with 2-sulfanylethanol and purified with a Sephacryl 300 column (available from Sigma).

Iron concentrations were determined by absorbance at 410 nm after one hour incubation in 6N HCl and H₂O₂ to dissolve the CLIO. Protein concentrations were determined by bicinchoninic acid assay (available from Pierce). The protein concentration was divided by the iron concentration to estimate the number of antibodies conjugated to each nanoparticle, assuming 8000 iron molecules per CLIO, as described in F. Reynolds et al, “Method of determining nanoparticle core weight,” Anal Chem 77 (2005) pp. 814-817, which is incorporated by reference in its entirety.

The anti-analytes to hCG, comprising monoclonal antibodies conjugated to CLIO nanoparticles are referred to by a shortened form of their product numbers, C95 and C97. The antibodies of C95 and C97 are a matched pair to separate, non-overlapping epitopes on hCG, with a K_{d} of 10⁻¹⁰ M and 5×10⁻⁹ M, respectively. In an additional embodiment, a second batch of anti-analytes, C95_{—}2 and C97_{—}2, was produced by a three-fold scale up of the reactants. C95_{—}2 and C97_{—}2 resulted in higher valencies than C95 and C97, yielding approximately 1 to 2 more antibodies per nanoparticle.

An average number of ligands, or valencies, per CLIO nanoparticle was determined as described above, and is reported in Table 1. Additionally, NMR transverse T₂ and longitudinal T₁ relaxation times were measured for conjugated and non-conjugated CLIO nanoparticles at various iron concentrations (mM). The relaxivities are reported in Table 1 as the slope of a plot of 1/T₂ or 1/T₁ (s⁻¹) as a function of the iron concentration.

TABLE 1
Characterization of Functionalized CLIO Nanoparticles

| | | R2 RELAXIVITY | R1 RELAXIVITY | VALENCY (No. |
|---|---|---|---|---|
| SAMPLE | ANALYTE | (mM sec)−1 | (mM sec)−1 | per particle) |
| CLIO | n/a | 54.2 | 21.53 | n/a |
| CLIO-IgG | protein A | 47.0 | n/m | 3.6 |
| CLIO-95 | hCG, hCG-β | 44.1 | n/m | 2.2 |
| CLIO-97 | hCG, hCG-β | 44.9 | n/m | 3.1 |
| CLIO-95_2 | hCG, hCG-β | 67.9 | 17.8 | 4.1 |
| CLIO-97_2 | hCG, hCG-β | 76.4 | 20.0 | 4.5 |

n/a: not applicable
n/m: not measured

### Example 3

### Measurements of Aggregation: NMR

In various embodiments, nuclear magnetic relaxation techniques are used to evaluate the aggregation of analytes and anti-analytes. The formation of aggregates within a region can alter one or both of the transverse T₂ or longitudinal T₁ relaxation times for an NMR-active element or isotope having non-zero nuclear magnetic spin within the region. For example, the hydrogen proton (H⁺) present in water is an NMR-active element which can provide a measurable NMR signal. Its transverse and longitudinal relaxation times can be affected by the presence of aggregates containing iron.

For various examples described herein, proton relaxation time measurements were performed on one of two instruments under slightly different conditions. One measurement process was carried out at 0.47 Tesla and 40° C. using a Bruker NMR Minispec (available form Bruker, Billerica, Mass.). In some cases, samples were incubated at 40° C. for one hour for thermal equilibration before measurements were taken. A second measurement process was carried out at 0.47 Tesla and 25° C. using an ex situ MR system (available from RWTH-Aachen, Aachen, Germany). The ex situ MR system was retrofitted with a programmable positioning stage for automated, high-throughput measurements. These instruments provided measurements of both transverse T₂ or longitudinal T₁ relaxation times. In various embodiments, measurements were made for a limited region 180 within the matrix 120, or within a solution for several non-stabilized aggregation trials reported herein. Longer-term stability measurements were obtained by measuring NMR relaxation times after periods of incubation at room temperature. Where error bars are shown, data are reported as average relaxation time, with error bars indicating plus and minus one standard deviation.

### Example 4

### Measurements of Aggregation: Optical

In some embodiments, optical techniques can be used to evaluate the aggregation of analytes and anti-analytes. The formation of aggregates within a volume of material can alter the optical transmission characteristics of the material, in terms of one or both of optical intensity and frequency. In some embodiments, the transmission characteristics can be used to determine analyte concentration and/or size of the aggregates.

As an example, particle sizes in solution, i.e., for a non-stabilized aggregation system, were determined using a 90 Plus Particle Size Analyzer (available from Brookhaven Instrument Corporation, Holtsville, N.Y.). Samples were measured at 37° C. in phosphate buffered saline, λ=658 nm, 90° fixed angle. The lognormal intensity-weighted effective diameter was determined from the measurement. Population distribution was determined by peak integration of the volume-weighted scattering intensity. Similar optical measurements can be made for aggregates suspended in a matrix, provided the matrix is substantially transparent to the incident optical radiation.

### Example 5

### Preparation of a Stabilizing Matrix from Agarose

In this example, a stabilizing matrix 120 having anti-analytes 130 dispersed therein was prepared from 1% agarose and the C95-C97 functionalized CLIO nanoparticles prepared as described in Example 2. The preparation of the matrix comprised the following steps:

(a) The 1% agarose was heated in a microwave oven to form a molten substance.

(b) A first volume of solution having a concentration of C95-C97 of about 16 μg/ml Fe was mixed with a second volume of the molten 1% agarose. In one embodiment, the first and second volumes were substantially equal, yielding a molten substance comprising about 0.5% agarose solution and about 8 μg/ml Fe concentration of C95-C97 anti-analytes.

(c) While still molten, the substance was pipetted into vessels. In one embodiment, the vessels were wells of a 96-well plate. The substance was pipetted into the wells in a manner to homogenize the substance within the well and avoid introduction of air bubbles into the substance. In some embodiments, any air bubbles inadvertently introduced into the substance could be removed by placing the vessels into a vacuum environment.

(d) The molten substance was left in the vessel for a period of time, to allow it to set, e.g., cool and substantially solidify. In various embodiments, the amount of time required for setting can vary, and depends in part on the gelling temperature of the agarose used.

### Example 6

### Non-Stabilized Aggregation System

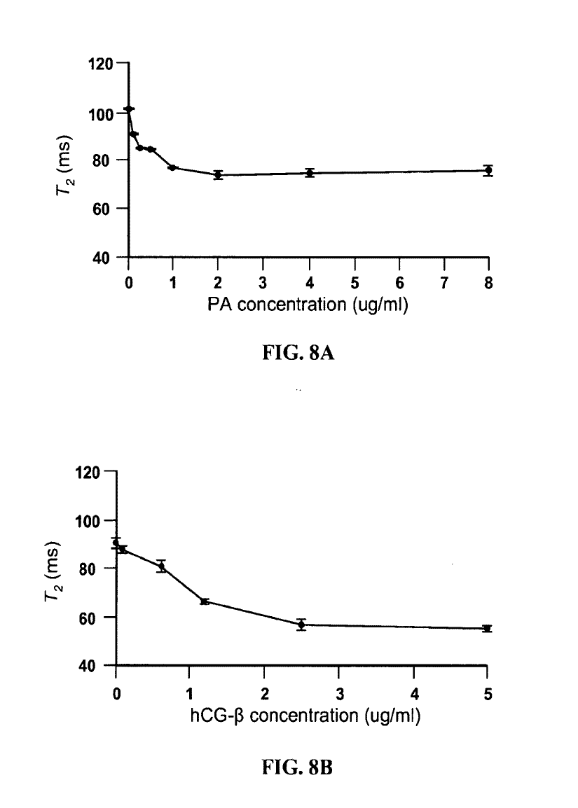

For purposes of comparison, several experiments were carried out with non-stabilized aggregation systems. Details of these experiments are reported in this example and in FIGS. 8A-8B, 9A, 10A, and 11A. Trials which generated the data reported in these figures were carried out without the use of a stabilizing matrix 120.

Non-stabilized aggregation experiments were performed by mixing approximately equal volumes of anti-analyte solutions and analyte solutions. In one trial, CLIO-IgG nanoparticles were mixed with various protein A (PA) concentrations. In another trial, an equal mixture of C95 and C97 (C95-C97) solution, or C95_{—}2 and C97_{—}2 (C95_{—}2-C97_{—}2) solution, was mixed with a solution containing hCG or hCG-β analytes. Analyte dilutions of PA (available from Sigma, 45 kDa) and hCG-β (available from Scripps Laboratories, 28 kDa) were prepared in phosphate buffered saline (PBS) pH 7.4 with 1% penicillinstreptomycin and 0.1% or 1% bovine serum albumin (in PBS) to reduce non-specific adsorptive loss of analyte. Reported concentrations were the final analyte concentrations obtained after mixing.

FIG. 8A-8B are plots showing the change in measured proton (H⁺) transverse relaxation time T₂ as a function of analyte concentration. For the aggregation system of FIG. 8A (mouse IgG:protein A in solution), the measured relaxation time reduces substantially nonlinearly from about 100 milliseconds (ms), with no analyte present, to about 72 ms at an analyte concentration of about 1.5 μg/ml. For analyte concentrations greater than about 2 μg/ml, there is substantially no change in the measured relaxation time. Above about 2 μg/ml, the T₂ signal is substantially saturated, so that further changes in analyte concentration produce substantially no change in the T₂ signal. Accordingly, an upper bound of the dynamic range for the aggregation system of FIG. 8A is about 2 μg/ml. A similar trend is observed for the double anti-analyte aggregation system (C95-C97:hCG-β in solution), as represented in FIG. 8B. For this case, analyte concentrations greater than about 2.5 μg/ml produce substantially no change in the proton transverse relaxation time T₂. An upper bound of the dynamic range for the aggregation system of FIG. 8B is about 2.5 μg/ml.

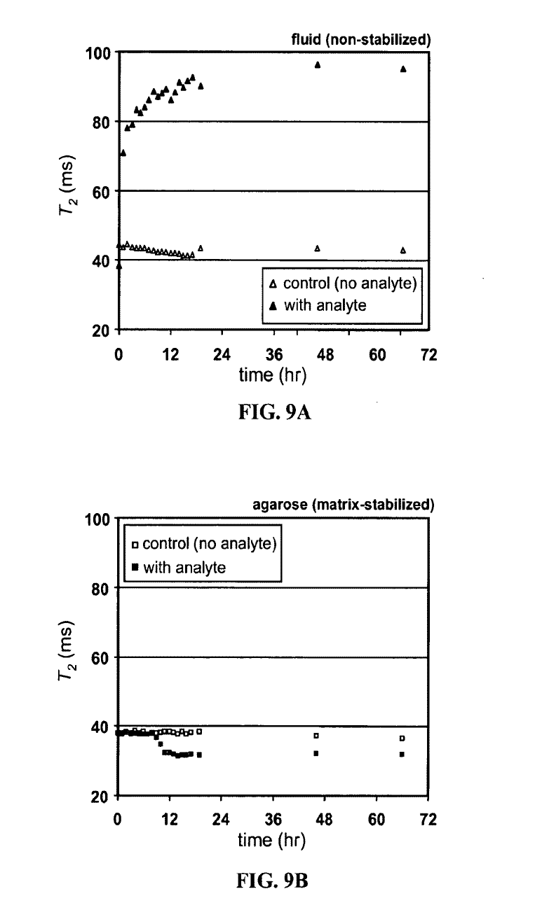

FIG. 9A is a plot of measured transverse relaxation times as a function of time interval elapsed after intermixing of a non-stabilized, double-anti-analyte, aggregation system (C95_{—}2-C97_{—}2:hCG in solution). hCG is a heterodimer, consisting of an alpha and beta subunit, and will also form aggregates with the matched pair anti-analytes C95_{—}2-C97_{—}2. For this trial, the analyte concentration was about 5 μg/ml hCG. The open triangles were measured in a control run, i.e., without the addition of the analyte. The filled triangles show that an initial value of about 40 ms, measured substantially immediately after mixing analytes and anti-analytes, increases rapidly to about 70 ms, and then drifts upward to about 100 ms over a period of about 48 hours. The measured signal, transverse relaxation time, is not stable for a period of about 20 hours after intermixing analytes and anti-analytes. The upward drift and increase in relaxation time is associated with precipitation of large, insoluble aggregates out of solution. The control measurement (no analyte, open triangles) shows a substantially stable value of T₂ (about 43 ms) measured over the same time period. The final anti-analyte concentration was about 8 μg/ml Fe CLIO nanoparticles for the aggregation system of FIG. 9A.

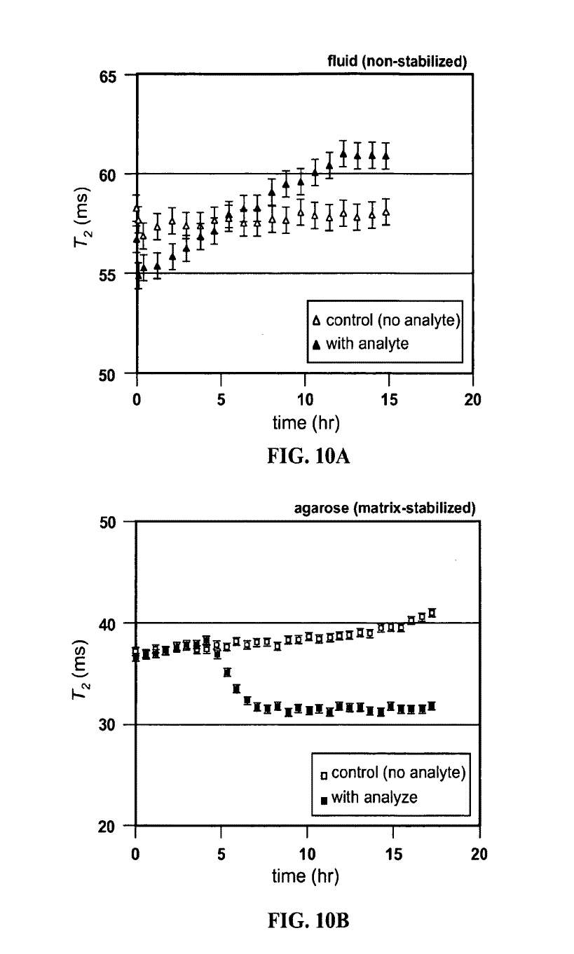

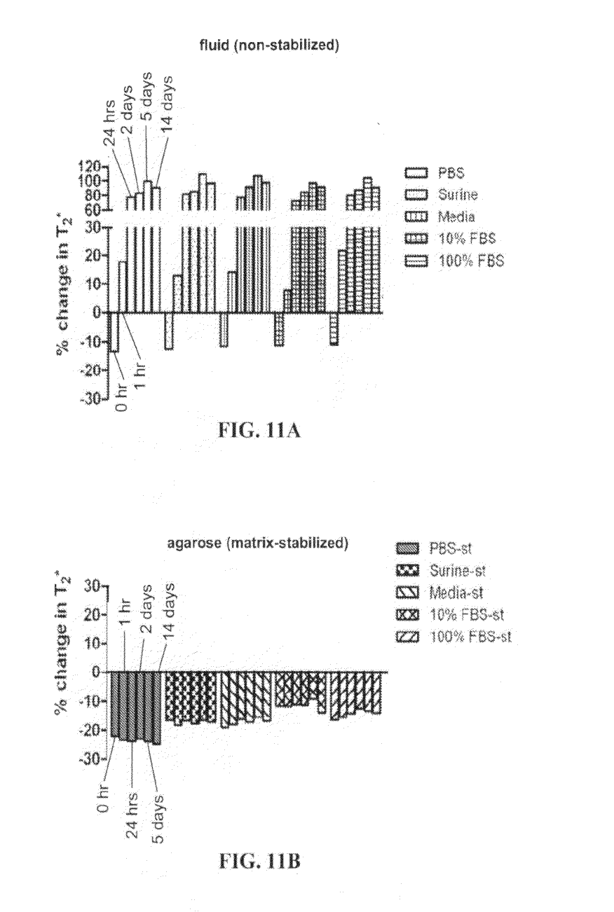

FIG. 10A shows a similar result to FIG. 9A. For this trial, the aggregation system is (C95-C97:hCG in solution), and the analyte concentration was about 1.25 μg/ml. The control measurement again shows a substantially stable value of T₂ (about 58 ms) over the duration of the measurement. Again, the measured value of T₂ drifts upward over the duration of the measurement, an unstable result. The reliability of non-stabilized aggregation systems can be significantly compromised by uncontrollable growth of aggregate products, and their precipitation out of solution. The final anti-analyte concentration was about 8 μg/ml Fe CLIO nanoparticles for the aggregation system of FIG. 10A.

The data plotted in FIG. 11A reports a percent change in an adjusted transverse relaxation time T₂* as a function of time for several different buffer solutions. For the measurements of FIGS. 11A-B, the ex situ MR instrument was used. The instrument's field gradient add a constant offset to

$$\frac{1}{{T}_{2}}$$

such that

$$\frac{1}{{T}_{2}^{*}} = \frac{1}{{T}_{2}} + \frac{1}{{T}_{2 , f}} \\ where \\ \frac{1}{{T}_{2 , f}} \qquad EQ . 1$$

is the contribution of the field gradient at a given temperature, pulse width and echo time. The buffers containing the analyte included were PBS, artificial urine solution (Surine®), cell culture media, cell culture media plus 10% fetal bovine serum (FBS), and 100% FBS. The analyte concentration in each buffer was about 2.5 μg/ml. It can be seen from FIG. 11A that each of the aggregation systems were unstable. Initial changes in T₂* reversed over time and drifted in value, similar to results associated with FIGS. 9A and 10A.

### Example 7

### Stabilized Aggregation Systems

In this example, various embodiments of matrix-stabilized aggregation systems are demonstrated. Several experiments were carried out to demonstrate stabilization of aggregation systems. The experiments use the double-anti-analytes C95-C97, or C95_{—}2-C97_{—}2 described in Example 2. The analyte for the experiments was hCG.

For the experiments, a stabilizing matrix containing a concentration of anti-analytes was prepared from an agarose mixture as described in Example 5. The molten substance was deposited into wells of 96-well plates. For one experiment (FIG. 9B), a concentration of hCG analyte of about 5 μg/ml hCG was dispensed into a vessel on top of the matrix. The matrix was about 4.5 mm thick in the wells, and the double anti-analytes were C95_{—}2-C97_{—}2. For a second experiment (FIG. 10B), a concentration of hCG analyte of about 5/ml was dispensed in a vessel on top of the matrix. The matrix thickness was about 3 mm thick in the wells, and the double anti analytes were C95_{—}2-C97_{—}2. For both experiments, NMR relaxometry measurements were made at multiple time intervals after the introduction of the analyte solution into the vessels. Control measurements were also made for each case, for which the same solution, having no analyte, was introduced to similar vessels. Results from the aggregation measurements (filled symbols) and control measurements (open symbols) are plotted in FIG. 9B and FIG. 10B.

The results plotted in FIG. 9B and FIG. 10B are similar: the control experiments show a substantially constant value of the proton transverse relaxation time over the duration of the measurement, and the aggregation measurements show a delayed decrease in relaxation time after which the measured value remains substantially constant or stable. For the aggregation system (C95_{—}2-C97_{—}2:hCG in matrix) of FIG. 9B, the T₂ time reduces from about 38 ms to about 31 ms between about 10 and about 12 hours after the introduction of the analyte. The measured value then remains substantially constant for at least about 60 hours. This can be compared with the non-stabilized case of FIG. 9A. The stabilization of the T₂ value can be attributed to the matrix inhibiting uncontrolled aggregation of the analytes and anti-analytes, and maintaining the aggregate products within the matrix.

Similar results were obtained for the aggregation system (C95-C97:hCG in matrix) of FIG. 10B. For the measurements of FIG. 10B, the gel thickness was about 3 mm, and the NMR measurement region was a horizontal slab about 250 microns thick and located at about mid height of the gel. For FIG. 10B the transition period where T₂ changes in value occurs about 6 hours after analytes in solution are added to the vessel. Because the thickness of the gel was reduced as compared to the case of FIG. 9B, the delayed reduction in the T₂ value occurs sooner, e.g., in about 6 hours compared to about 10 hours. The concentration of the hCG analyte (5 μg/ml) used in this trial was much higher than can be tolerated with non-stabilized, liquid, CLIO-based aggregation systems. At such high analyte concentrations, uncontrolled aggregation and precipitation of products occurs in the entirely aqueous solutions.

The onset of the T₂ signal transition period in the aggregation measurements of FIG. 9B and FIG. 10B is attributed to the diffusion of hCG into the measurement volume. In various embodiments, this time-delayed reduction can be manipulated by a variety of means, including but not limited to: diffusion length of the analyte into the matrix, matrix thickness, matrix density, matrix porosity, temperature, electrophoresis, cross-linking of polymeric material within the matrix, and viscosity of the analyte solution. Additionally, the time delay can be shortened by measuring a region 180 within the stabilizing matrix located closer to a surface which contacts the analyte solution, e.g., near the upper surface of the material 120 as depicted in FIG. 1D.

### Example 8

### Measurements of Analyte Concentration: Dynamic Range and Stability

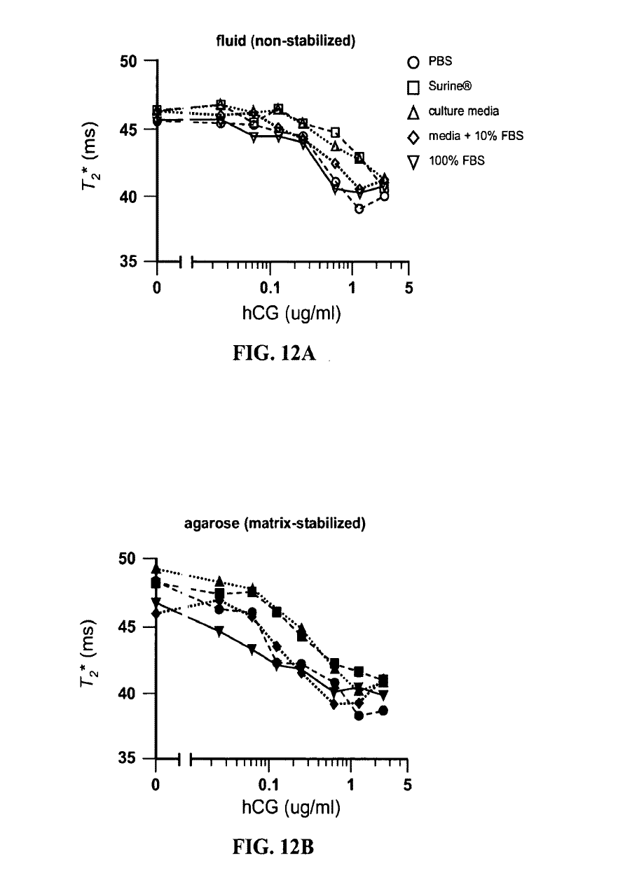

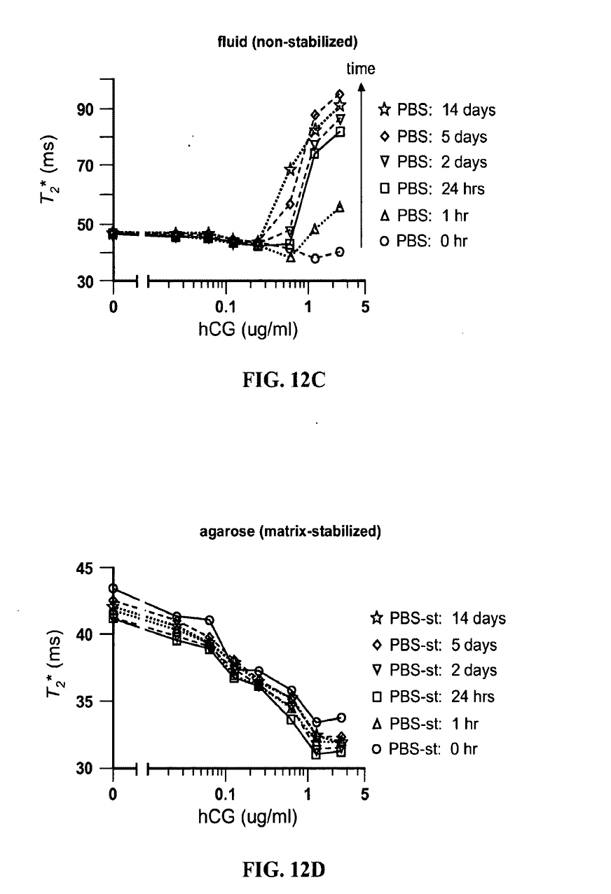

Several experiments were carried out to assess and compare dynamic range and stability for various aggregation systems. The results are reported in FIGS. 12A-D. The results show that matrix-stabilized agglutination systems can provide significantly increased stable dynamic range as compared to non-stabilized agglutination systems.

Changes in measured values of T₂* for various analyte concentrations in several different buffers, and for several different incubation periods, are reported in FIGS. 12A-D. In FIGS. 12A-B, five different buffers were used: phosphate buffered saline (PBS) artificial urine (Surine), tissue culture media, media supplemented with 10% fetal bovine serum (FBS), and 100% FBS. For FIG. 12A, the aggregation-based assays were carried out in liquid and not stabilized. For FIG. 12B, the aggregation-based assays included a stabilizing agarose matrix. In FIGS. 12C-12D, the stability of two of the aggregation systems was assessed: PBS buffer and non-stabilized aggregation (FIG. 12C), and PBS buffer and matrix-stabilized aggregation (FIG. 12D). The data of FIG. 12B indicates an increase in the signal dynamic range for the stabilized aggregation systems, as compared to the non-stabilized case (FIG. 12Ab). The effect is most noticeable at the lower bound of the dynamic range, i.e., lower analyte concentration. The matrix-stabilized aggregation-based assays indicate less saturation of the low-concentration signal. The improvement in stable dynamic range is particularly evident in the data of FIGS. 12C-D. The observed dynamic range for the matrix-stabilized system, FIG. 12D, remains stable for at least 14 days. This is not true for the non-stabilized system, FIG. 12C, where the initially measured dynamic range of FIG. 12A becomes unstable for analyte concentrations greater than about 0.25 μg/ml. For analyte concentrations exceeding this value in non-stabilized systems, the measured signal can become unreliable soon after the aggregation test is carried out. A comparison of the data in FIGS. 12C-D indicates that matrix-stabilized aggregation-based assays can provide stable dynamic ranges about an order of magnitude greater than non-stabilized agglutination systems. For example, the data of FIG. 12C indicates a non-stabilized dynamic range for the particular aggregation assay extending from about 0.025 mg/ml to about 0.25 μg/ml, a change in analyte concentration by about a factor of 10. The data of FIG. 12D indicates a matrix-stabilized dynamic range extending from less than about 0.02 μg/ml to greater than about 1.0 μg/ml, a change in analyte concentration by about a factor of 500. In some embodiments, matrix-stabilized aggregation assays provide stable dynamic ranges over a change in analyte concentration by greater than about a factor of 10, greater than about a factor of 20, greater than about a factor of 50, greater than about a factor of 100, greater than about a factor of 200, and yet in some embodiments greater than about a factor of 500.

For the data reported in FIG. 12B and FIG. 12D, the aggregation constituents were mixed together in the matrix material while the matrix was in a molten or flowable state. The mixture was then dispensed in wells of a multi-well plate, and allowed to set for approximately 5 minutes. Measurements were then made using the ex situ MR instrument.

### Example 9

### High-Throughput Measurements

When collecting data for the plots of FIGS. 12A-D, it was necessary to measure a large number of samples, e.g., 96 samples from a 96-well plate. To accomplish multiple readings, a high-throughput MR measurement system was developed. The ex situ MR instrument from RWTH-Aachen was retrofitted with a programmable positioning stage for automated, high-throughput measurements. In various embodiments, the positioning stage is programmed to position sequentially each of the 96 wells within a measurement area of the ex situ MR instrument, so that at least a portion of the matrix may be probed to detect a signal representative of an amount of aggregation within the matrix. For the selected operating parameters, approximately 30 minutes were required to read an entire 96-well plate.

Since high-throughput measurements can require extended periods of time, e.g., about 30 minutes for a 96-well plate, or about 120 minutes for a 384-well plate, stability of the aggregation system, and aggregation signal, during the measurement period is desired. Matrix-stabilized aggregation systems can provide such stability in excess of 120 minutes, as indicated in FIG. 11B. Matrix-stabilized aggregation-based systems are therefore useful for high-throughput agglutination assays. In various embodiments, matrix-stabilized aggregation systems provide stable dynamic ranges for high-throughput aggregation assays.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. For example, although the present teachings describe aggregation systems in which one or more anti-analyte components are dispersed within a matrix and analytes in solution are brought into contact with the matrix containing anti-analytes, additional embodiments can include a matrix having analytes dispersed therein and bringing a solution containing anti-analytes into contact with the matrix containing analytes.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed is:

1. A method for quantitatively determining a concentration of analyte in a test solution, the method comprising steps of:
providing a matrix, the matrix comprising a substance having a viscosity greater than about 1.5 centipoise and anti-analytes, the anti-analytes dispersed within the substance and mobile within the substance;
contacting a test solution containing a concentration of analytes to the matrix so that the analytes permeate through at least a portion of the matrix; and
detecting a signal representative of an amount of aggregates that form within a volume of the matrix, the aggregates forming from the anti-analytes and the analytes, wherein
aggregates larger than a certain size are substantially immobile within the matrix.

2. The method of claim 1, where the step of providing further comprises providing an amount of matrix with anti-analytes in a vessel.

3. The method of claim 2, wherein the vessel has an optically-transparent portion through which light may enter and exit the vessel without significant scattering or attenuation of the light by the vessel.

4. The method of claim 2, wherein the vessel comprises a well of a multi-well plate.

5. The method of claim 2, wherein the vessel is adapted for pressurization of its contents.

6. The method of claim 2, wherein the vessel comprises a rounded depression formed in a polymer.

7. The method of claim 2, wherein the vessel is adapted for in vivo placement.

8. The method of claim 7, wherein the vessel is round, elliptical, or oblong with rounded features.

9. The method of claim 7, wherein the vessel further comprises a porous or semi-porous portion through which a solution and analytes native to the in vivo environment may flow.

10. The method of claim 1, where the step of providing further comprises providing similar amounts of matrix with anti-analytes in an array of vessels.

11. The method of claim 1, where the step of providing further comprises providing similar amounts of matrix with anti-analytes in an array of microdots on a microtitre plate.

12. The method of claim 1, where the step of providing further comprises providing similar amounts of matrix with anti-analytes in an array of depositions on a substrate.

13. The method of claim 1, where the step of providing further comprises providing the matrix with anti-analytes in a film on a substrate.

14. The method of claim 1, wherein the matrix comprises a plurality of beads.

15. The method of claim 14, wherein the beads have diameters in a range between about 50 nanometers and about 250 microns.

16. The method of claim 14, wherein the beads are formed from a polymer.

17. The method of claim 14, wherein the plurality of beads are held within a vessel.

18. The method of claim 1, wherein the matrix comprises a liquid.

19. The method of claim 18, wherein the viscosity of the liquid is greater than about 2 centipoise.

20. The method of claim 1, wherein the Brownian diffusion distance traveled by aggregates having a certain range of sizes during a time interval within the matrix is greater than the distance traveled by the aggregates due to gravitational forces for the same time interval.

21. The method of claim 1, wherein the matrix comprises a solid.

22. The method of claim 1, wherein the matrix comprises a gel or hydrogel.

23. The method of claim 1, wherein the matrix comprises a substance selected from the group consisting of: agarose gel, acrylamide, polyacrylamide, cellulose, chitosan, dextran, ficoll, silica gel, and any combination thereof.

24. The method of claim 1, wherein the matrix comprises a polymer.

25. The method of claim 24, wherein the matrix comprises a polymer selected from the group consisting of: methacrylate, polystyrene, polyvinylalcohol, polyethyleneglycol, polyurethane, polycarbonate, polyarylate, polymethylmethacrylate, and any combination thereof.

26. The method of claim 1, wherein the matrix comprises a porous ceramic material.

27. The method of claim 26, wherein the porous ceramic material is selected from the group consisting of: ceramic colloidal gels, ceramic fiber meshes, ceramic colloidal particles, sintered ceramic beads, and any combination thereof.

28. The method of claim 1, wherein the matrix comprises a mesh of glass fibers.

29. The method of claim 1, wherein the matrix comprises cellulose.

30. The method of claim 1, wherein the matrix comprises a porous scaffold or inverse opal scaffold.

31. The method of claim 1, wherein the matrix comprises a substance which is substantially solid or solid at about room temperature and flowable when heated to a temperature above room temperature.

32. The method of claim 1, wherein the matrix is biocompatible and/or biodegradable.

33. The method of claim 1, wherein the matrix has a mean intrinsic pore size between about 50 nanometers and about 500 microns.

34. The method of claim 33, wherein the distribution of pore sizes within the matrix is between about 10% and about 100% of the average pore size.

35. The method of claim 1, where the step of providing further includes selecting the matrix according to its mean intrinsic pore size.

36. The method of claim 1, wherein the matrix expands upon absorption of a liquid.

37. The method of claim 1, wherein the matrix is transformable into a molten or flowable state and subsequently transformable into a substantially solid or semi-solid or gel state.

38. The method of claim 37, wherein the matrix is transformable into a molten or flowable state by heating the matrix.

39. The method of claim 37, wherein the matrix is transformable into a molten or flowable state by adding a solvent to the matrix.

40. The method of claim 37, wherein the matrix is transformable into a substantially solid or semi-solid or gel state by cooling the matrix.

41. The method of claim 37, wherein the matrix is transformable into a substantially solid or semi-solid or gel state by exposing the matrix to ultraviolet radiation.

42. The method of claim 37, wherein the matrix is transformable into a substantially solid or semi-solid or gel state by adding a chemical cross-linking agent to the matrix.

43. The method of claim 37, wherein the matrix is transformable into a substantially solid or semi-solid or gel state by providing heat to cross-link the matrix.

44. The method of claim 1, wherein the anti-analytes were dispersed within the matrix by mixing a solution containing the anti-analytes into the matrix while the matrix was in a molten or flowable state.

45. The method of claim 1, wherein the anti-analytes were dispersed within the matrix by immersing the matrix in a solution containing a concentration of anti-analytes.

46. The method of claim 1, wherein the matrix was stored prior to use in a solution containing a concentration of anti-analytes.

47. The method of claim 1, wherein the matrix incorporates topography so as to increase the surface area of the matrix.

48. The method of claim 1, wherein the matrix is hydrophilic.

49. The method of claim 1, wherein the anti-analytes comprise particles having similar diameters of a value between about 10 nm and about 250 microns.

50. The method of claim 1, wherein the anti-analytes comprise antibodies that bind to the analyte.

51. The method of claim 1, wherein the anti-analytes comprise two different types of anti-analytes.

52. The method of claim 51, wherein
a first type of anti-analyte comprises a chemically functionalized nanoparticle having a first type of ligand which binds to a first type of receptor on the analyte; and
a second type of anti-analyte comprises a chemically functionalized nanoparticle having a second type of ligand which binds to a second type of receptor on the analyte.

53. The method of claim 51, wherein both types of anti-analytes comprise a reporter.

54. The method of claim 53, wherein the reporter comprises a cross-linked iron-oxide nanoparticle.

55. The method of claim 53, wherein the reporter comprises an isotope having non-zero nuclear magnetic spin.

56. The method of claim 53, wherein the reporter comprises a ligand.

57. The method of claim 53, wherein the reporter comprises a fluorescent molecule.

58. The method of claim 1, wherein the step of detecting comprises detecting signals provided by, or altered by, reporters present with the aggregates.

59. The method of claim 58, wherein the reporters comprise cross-linked iron-oxide nanoparticles.

60. The method of claim 58, wherein the reporters comprise isotopes having non-zero nuclear magnetic spin.

61. The method of claim 58, wherein the reporters comprise ligands.

62. The method of claim 58, wherein the reporters comprise fluorescent molecules.

63. The method of claim 58, wherein the signals are detected by nuclear magnetic resonance.

64. The method of claim 58, wherein the signals are detected by optical detection.

65. The method of claim 1, wherein the analyte is a protein.

66. The method of claim 1, wherein the analyte is a cell.

67. The method of claim 1, wherein the analyte is a molecule.

68. The method of claim 1, wherein the analyte is a virus.

69. The method of claim 1, wherein the analyte is a portion of DNA.

70. The method of claim 1, wherein the analyte has multiple types of anti-analyte binding sites.

71. The method of claim 1, further comprising between the step of contacting and the step of detecting:
providing an amount of time for the permeation of analytes through a volume of the matrix.

72. The method of claim 1, wherein the aggregates become substantially immobile in the matrix when the size of an aggregate is greater than a value between about 100 nm and about 2000 nm.

73. The method of claim 1, wherein the aggregates remain substantially lodged at their location within the matrix for a duration of time exceeding about 30 minutes.

74. The method of claim 1, where the step of providing further comprises selecting a matrix by its mean intrinsic pore size to limit the maximum size of aggregates mobile within the matrix.

75. The method of claim 1, wherein the step of contacting further comprises increasing mobility of the analytes or anti-analytes within the matrix using a technique selected from the group consisting of increasing pressure in a region containing the matrix, applying electric fields within the matrix, applying magnetic fields within the matrix, applying centrifugal force to the matrix, applying ultrasonic agitation to the matrix, heating the matrix, shaking the matrix, and any combination thereof.

76. The method of claim 1, wherein the step of measuring comprises detecting a signal representative of an amount of aggregation within a measurement volume of the matrix.

77. The method of claim 76, wherein the measurement volume comprises a slab-like section of the matrix.

78. The method of claim 76, wherein the measurement volume comprises a shape selected from the group consisting of a cube, rectangle, sphere, or oblate sphere.

79. The method of claim 76, wherein the measurement volume comprises substantially the entire volume occupied by the matrix.

80. The method of claim 1, where the step of providing further comprises providing a plurality of matrices with anti-analytes, the matrices having different mean intrinsic pore sizes and/or anti-analyte concentrations.

81. The method of claim 1, wherein the step of providing comprises:
mixing a molten matrix with a plurality of anti-analytes to form a molten mixture;
dispensing an amount of the molten mixture into a vessel or onto a substrate; and
allowing the molten mixture to set.

82. The method of claim 1, where the step of providing further comprises placing a vessel containing an amount of matrix and anti-analytes in vivo.

83. The method of claim 1, further comprising:
recording the amount of the matrix;
recording the concentration of anti-analytes within the matrix; and
recording the amount of test solution containing analytes contacted to the matrix and anti-analytes.

84. The method of claim 1, where the step of detecting further comprises:
measuring a value of the detected signal;
comparing the measured value with values obtained from calibration standards; and
determining a concentration of the analyte in the test solution based on the comparison.

85. The method of claim 1, where the step of detecting is carried out by a technique selected from the group consisting of: nuclear-magnetic-resonance imaging, nuclear-magnetic-resonance spectroscopy, nuclear magnetic relaxometry, optical scattering, optical spectroscopy, optical imaging, optical fluorescence detection, infrared imaging, infrared spectroscopy, infrared scattering, and x-ray imaging, and any combination thereof.

86. The method of claim 1, wherein the signal representative of an amount of aggregates is stable for a period of time greater than about 30 minutes.

87. The method of claim 86, wherein the signal varies by less than ±25% during the period for which the signal is stable.

88. The method of claim 1, where the step of providing further comprises providing a list or data bank of calibration values and associated analyte concentrations with the matrix.

89. The method of claim 1, where the step of contacting comprises:
placing the test solution into contact with the surface of the matrix.

90. The method of claim 1, where the step of contacting comprises:
rendering the matrix in a molten state;
adding the test solution to the molten matrix;
mixing the test solution and molten matrix; and
allowing the mixture to set.

91. The method of claim 1, further comprising repeating the method with a different analyte concentration so that the signal representative of an amount of aggregates falls within a dynamic range for the matrix with anti-analytes.

92. The method of claim 1, where the step of providing further comprises providing information about the dynamic range of the matrix with anti-analytes dispersed therein.

93. The method of claim 1, wherein the matrix with anti-analytes dispersed therein has a dynamic range which is stable for a period of time exceeding about 1 hour.

94. The method of claim 93, wherein the dynamic range is greater than about a factor of 10.

95. An apparatus for aggregation-based assays comprising:
an amount of matrix and a known concentration of anti-analytes, wherein:
the matrix comprises a substance having a viscosity greater than about 1.5 centipoise;
the anti-analytes are dispersed within the matrix;
the anti-analytes and analytes are mobile within the matrix; and
aggregates of anti-analytes and analytes larger than a certain size are substantially immobile within the matrix.

96. The apparatus of claim 95 further comprising:
a vessel, the vessel containing an amount of the matrix with anti-analytes.

97. The apparatus of claim 96 wherein the vessel is adapted for in vivo placement.

98. The apparatus of claim 95 further comprising:
a multi-well plate, one or more wells of the multi-well plate containing an amount of the matrix with anti-analytes.

99. The apparatus of claim 95 further comprising:
a microtitre plate including one or more deposits of an amount of matrix with anti-analytes.

100. A method for quantitatively determining a concentration of analyte in a test solution, the method comprising steps of
providing a matrix, the matrix comprising a substance having a viscosity greater than about 1.5 centipoise and anti-analyte aggregates dispersed within the substance, the anti-analyte aggregates being substantially immobile within the substance;
contacting a test solution containing a concentration of analytes to the matrix so that the analytes permeate through at least a portion of the matrix; and
detecting a signal representative of an amount of aggregates that form within a volume of the matrix, wherein
the aggregates dissipate upon interaction with the analytes.
